(19) United States
(12) Patent Application Publication
(10) Pub. No.: US 2020/0135334 A1
(43) Pub. Date: Apr. 30, 2020

(54) DEVICES AND METHODS FOR REMOTELY MANAGING CHRONIC MEDICAL CONDITIONS

(71) Applicant: AIRx Health, Inc., San Jose, CA (US)

(72) Inventors: Vijaykumar RAJASEKHAR, Apple Valley, CA (US); Marlon Sebastian CASTILLO, San Mateo, CA (US); Ritesh Narayan RAGAVENDER, San Francisco, CA (US); Frank James BUNCOM, IV, San Diego, CA (US)

(21) Appl. No.: 16/664,434

(22) Filed: Oct. 25, 2019

Related U.S. Application Data
(60) Provisional application No. 62/849,008, filed on May 16, 2019.
(60) Provisional application No. 62/750,819, filed on Oct. 26, 2018.

(51) Int. Cl. G16H 40/67; G06N 3/00; G10L 15/22; G10L 15/26; G08B 25/01; H04M 3/42; H04M 11/04; G08B 21/04; H04M 3/493; A61B 5/00; A61B 5/0205

(52) U.S. Cl. CPC G16H 40/67; A61B 5/024; G10L 15/22; G10L 15/265; G08B 25/012; H04M 3/42221; H04M 3/42008; H04M 11/045; G08B 21/0453; H04M 3/4936; A61B 5/0022; A61B 5/7282; A61B 5/746; A61B 5/0205; H04M 2201/40; G06N 3/006

(57) ABSTRACT

A method for managing or remotely monitoring chronic medical conditions (e.g., chronic respiratory conditions) of a plurality of patients includes, at one or more processors, receiving a plurality of bodily metrics associated with each of the plurality of patients over one or more remote communication links (e.g., an automated phone system), characterizing the severity of the medical condition for each patient based on the plurality of bodily metrics associated with the patient, and transmitting a programmed alert to a medical care provider for at last one patient having a medical condition characterized as having a threshold level of severity.

### CROSS-REFERENCE TO RELATED APPLICATIONS

This application claims priority to U.S. Provisional Application Ser. No. 62/849,008 filed May 16, 2019, and U.S. Provisional Application Ser. No. 62/750,819 filed Oct. 26, 2018, each of which is hereby incorporated herein by reference in its entirety.

### TECHNICAL FIELD

This invention relates generally to the field of medical treatment of chronic medical conditions, and more specifically to devices and methods for remotely managing patients with chronic medical conditions.

### BACKGROUND

Millions of patients suffer from chronic medical conditions, including chronic respiratory conditions such as chronic obstructive pulmonary disease (COPD), asthma, and sleep apnea. For example, COPD is a lung disease characterized by chronic obstruction of lung airflow that interferes with normal breathing and is not fully reversible. COPD is a common, deadly, and expensive medical condition impacting approximately 24 million patients in the U.S., and is estimated to impact over 300 million patients globally. COPD is the third leading cause of death both globally and in the U.S. The 5-year mortality for COPD ranges from 40%-70% and is the only major cause of death on the rise globally. The cost of COPD is approaching $50 billion in the U.S. alone. A large portion of healthcare cost of COPD is due to frequent hospitalizations of patients. 50% of COPD patients are readmitted within 6 months, and 1 in 5 patients are readmitted within 30 days. COPD is predicted to become the #1 cause of hospitalizations within 5 years in the U.S.

COPD patients have diminished lung function. Small triggers, such as allergies, smoke, viral or bacterial infections, can increase secretions and/or limit lung function, further closing the airways. Such episodes are called COPD exacerbations, or COPD flares. Medications such as beta-agonists, anticholinergics, steroids, and antibiotics can help manage and/or prevent the symptoms of COPD exacerbations.

Currently there are limited options to monitor the signs and symptoms of COPD patients when they are not in the hospital setting. This limitation to identify patients at risk of deterioration in the outpatient setting frequently leads to delay in dose modification and titration that could otherwise prevent COPD exacerbations. Thus, today, many patients with COPD frequently visit the emergency room and are hospitalized due to acute episodes of shortness of breath. While in the hospital, patients receive escalating doses or additional classes of medications via inhalers, nebulizers, as well as oral/IV steroids and/or oral/IV antibiotics, and supplemental oxygen when needed. If patients arrive in the hospital with very severe difficulty breathing, they may require intubation with a breathing tube and ventilator with admission to the Intensive Care Unit. Patients stay in the hospital until the symptoms improve and are typically discharged with a modified care plan with adjusted medications, pulmonary rehabilitation, and/or change in supplemental home oxygen.

Furthermore, in the U.S., the ratio of COPD adults to pulmonologists ranges from 200:1 to as high as 3000:1 depending on the county. Thus, there is currently a shortage of pulmonologists who can appropriately manage the millions of patients with COPD and other respiratory conditions.

Thus, there is a need for new and improved systems and methods for managing patients with chronic medical conditions.

### SUMMARY

Generally, a method for remotely monitoring chronic medical conditions of a plurality of patients may include, at one or more processors, receiving a plurality of bodily metrics (e.g., physiologic parameters) associated with each of a plurality of patients over one or more remote communication links (e.g., over an automated phone system), characterizing the severity of the medical condition for each patient based on the plurality of bodily metrics associated with the patient, and transmitting a programmed alert to a medical care provider for at least one patient having a medical condition characterized as having a threshold level of severity. In some variations, the method is performed to remotely monitor chronic respiratory conditions, and the method may include characterizing the severity of the chronic respiratory condition for one or more patients based on the plurality of bodily metrics associated with the one or more patients, and transmitting a programmed alert to a medical care provider for at least one patient having a respiratory condition characterized as having a threshold level of severity. Furthermore, in some variations, the method may include enabling the medical care provider to adjust a medical treatment for at least one patient in response to the transmitted programed alert.

In some variations, receiving the plurality of bodily metrics includes prompting at least one patient during a phone call established with an automated phone system to provide at least a portion of the plurality of bodily metrics. The automated phone system may, for example, be implemented with or otherwise configured using a programmable communications platform (e.g., a programmable voice service and/or a programmable text service). The one or more bodily metrics may be recited by the patient over the automated phone system, and the recited bodily metrics may be received and recorded. In these variations, the method may further include performing speech-to-text transcription of the recordings. Additionally or alternatively, one or more of the bodily metrics may be entered through a telephone keypad interface (e.g., touchtone dialing, virtual keypad on a display of a mobile electronic device, etc.). In some variations, the automated phone system may be configured to facilitate interactive communication between the medical care provider and at least one patient. In some variations, the method may include receiving the phone call through the automated phone system from a patient phone number associated with the at least one patient, and masking the patient phone number with a second phone number to de-identify the at least one patient. In some variations, characterizing the severity of the medical condition (e.g., respiratory condition) may include assessing at least a portion of the received bodily metrics substantially in real-time during the phone call.

In some variations, the method may include assessing the received bodily metrics, dynamically generating one or more supplemental prompts based on the assessment of at least one received bodily metric, and prompting at least one patient with the one or more supplemental prompts (e.g., follow-up questions) over the automated phone system to provide supplemental patient data in response to the one or more supplemental prompts. For example, one or more supplemental prompts may be generated based on at least one received bodily metric satisfying at least one predetermined dynamic threshold (e.g., a minimum value and/or a maximum value). In some variations, the at least one predetermined dynamic threshold may be customizable with respect to a medical care provider, the at least one patient, or both.

Characterizing the severity of the respiratory condition may be based at least in part on comparing one or more bodily metrics to one or more predetermined thresholds In some variations, characterizing the severity of the medical condition (e.g., respiratory condition) may include comparing at least a first bodily metric to a first predetermined threshold. Multiple predetermined thresholds, such as for multiple bodily metrics, may be used to characterize the severity of the medical condition. For example, the severity of the medical condition may be based at least in part on comparing a first bodily metric to a first predetermined threshold, and/or comparing a second bodily metric to a second predetermined threshold. Such predetermined thresholds may be customizable with respect to a medical care provider, one or more patients, or both.

In some variations, the plurality of bodily metrics may be received on a repeated basis, such as at least one daily over a monitoring period of two or more days (e.g., 16 days per calendar month). The method may include contacting at least one of a patient and a patient-designated contact person for that patient, in response to failing to receive the plurality of bodily metrics for the patient within a predetermined time window.

In some variations, the medical care provider may be associated with a virtual clinic. In some variations, the medical care provider may include an auxiliary staff member working under supervision of a second medical care provider, wherein the first medical care provider and the second medical care provider are working in different physical locations, at different times, or both Additionally or alternatively, the medical care provider may provide designated medical care coverage (e.g., emergency or backup care) during a covering period in the absence of a primary medical care provider associated with at least one patient, even if a prior patient-medical care provider relationship has not been established between the medical care provider providing coverage and the at least one patient.

The method may include establishing a patient-medical care provider relationship for at least one of the plurality of patients through an asynchronous store and forward transmission of medical information associated with the at least one patient, from an originating site to the medical care provider not at the originating site, and without a real-time presence of the patient. In some variations, the method may include verifying an identity of the at least one patient through a non-documentary identity verification process. In some variations, the method may include receiving a dynamic intake questionnaire to be reviewed by the medical care provider (e.g., questions may dynamically change in response to responses to previous questions in the questionnaire).

In some variations, at least one of the chronic medical conditions is a chronic respiratory condition. In these variations, the plurality of bodily metrics may include at least one of oxygen saturation, heart rate, breathlessness severity, cough severity, and mucus severity for the at least one patient. In some variations, receiving the plurality of bodily metrics may include receiving and recording a phone call during which one or more bodily metrics is recited over an automated phone system, wherein the plurality of bodily metrics further includes respiratory rate, and the method may include measuring respiratory rate at least in part by counting patient breaths detected in the recording. In some variations, the plurality of bodily metrics may include quantification of home supplemental oxygen used by the at least one patient (e.g., flow rate (e.g., L/min), or information relating to the same (e.g., volume, etc.).

In some variations, characterizing the severity of the medical condition (e.g., respiratory condition) may include deriving a first points value for each bodily metric, In some variations, receiving a plurality of bodily metrics for a patient may be repeated over a monitoring period, and characterizing the severity of the respiratory condition may include deriving a second points value of at least one bodily metric based on the relative change of the at least one bodily metric during the monitoring period, and generating an index score based at least in part on the first points values for the plurality of bodily metrics and the second points value of at least one bodily metric.

In some variations, the method may further include generating an index score for each patient, wherein the index score corresponds to the characterized severity of the patient, and further comprising sorting the plurality of patients according to the index scores for the plurality of patients. For example, sorting the plurality of patients may include grouping each of the plurality of patients into one of a low-risk category, a medium-risk category, and a high-risk category (or any suitable number and range of categories). Furthermore, in this example, transmitting a programmed alert to a medical care provider for at least one patient may include transmitting a programmed alert of a first type for at least one patient in the medium-risk category, and transmitting a programmed alert of a second type for at least one patient in the high-risk category.

In some variations, the method may include providing a reminder to the at least one patient over the automated phone system or other remote communication link to conform to prescribed medical treatment (e.g., a reminder to the patient to take his or her medication), and/or inviting the at least one patient to ask a question to the medical care provider (e.g., inviting the at least one patient to ask any questions relating to a prescribed medication or other aspect of a prescribed medical treatment).

Generally, in some variations, a method for managing a chronic respiratory condition includes, at one or more processors, receiving a plurality of bodily metrics of a remote communication link provided by a patient over an automated phone system, and characterizing the risk of an upcoming clinical event for the patient based on the received bodily metrics. For example, the patient may call into the automated phone system and provide various bodily metric values. The method may further include prompting a designated contact in response to the patient failing to provide at least one of the plurality of bodily metrics. In some variations, the plurality of bodily metrics is received at least once daily over a monitoring period of two or more days. The plurality of bodily metrics may include, for example, at least one of oxygen saturation, heart rate, breathlessness severity, cough severity, and sputum/mucus severity. Additionally or alternatively, the plurality of bodily metrics further comprises at least one of vocal quality, respiratory rate, and respiratory sounds. For example, respiratory rate and/or respiratory sounds may be derived from breath sounds and/or duration of phone call while the patient is on the phone after calling into the automated phone system. In some variations, the method may further include recording and processing a reference speech excerpt recited by the patient during a phone call to the automated phone system. For example, respiratory rate and/or respiratory sounds may be derived from the recorded reference speech excerpt. Furthermore, in some variations, the method may include verifying at least one of the received plurality of bodily metrics in response to the at least one bodily metric being outside of a predetermined range (e.g., an expected range based on historical data, patient characteristics, etc.) and thus suspected of being erroneous. Verifying the at least one received bodily metric may include, for example, prompting a manual review of the at least one received bodily metric to correct or confirm the patient data (e.g., prior to transmitting the patient data to a healthcare team, etc.).

Generally, in some variations, a method for managing a chronic respiratory condition of a patient includes remotely monitoring a patient by receiving patient status assessments (e.g., periodically) via a remote communication link, wherein the patient status assessments comprise a plurality of bodily metrics associated with the respiratory condition, characterizing risk of an upcoming clinical event for the patient based on the patient status assessments, and adjusting a medical treatment for the patient in response to the characterized risk of an upcoming clinical event. A variety of patient demographics may be managed, including patients diagnosed with chronic obstructive pulmonary disease (COPD). Furthermore, the remotely monitored patient may or may not have been previously hospitalized for COPD. In some variations, adjusting a medical treatment may include modifying dose or frequency of one or more of the following: short-acting beta agonists, short-acting muscarinic antagonists, long-acting beta agonists, long-acting muscarinic antagonists, inhaled corticosteroids, antibiotics, supplemental oxygen, nebulizer treatment, and pulmonary rehabilitation, or any suitable treatment adjustment.

Generally, in some variations, a system for remotely managing a chronic respiratory condition may include a measurement device including one or more sensors for measuring one or more bodily metrics of the patient, wherein the one or more sensors include a pulse oximeter for measuring at least one of oxygen saturation and heart rate, a docking station configured to removably receive the measurement device. At least one of the measurement device and the docking station may include a wireless communication system configured to transmit the one or more bodily metrics of the patient. In some variations, the measurement device may be a wearable device (e.g., pendant, patch, cuff, etc.). In some variations, the measurement device and/or the docking station may include a microphone and/or at least one of an audible notification and a visual notification. Furthermore, in some variations, the docking station may include a power supply to recharge a power source of the measurement device.

In some variations, a wearable device for remotely managing a chronic respiratory condition of a patient includes a housing, one or more sensors for measuring one or more bodily metrics of the patient, wherein the one or more sensors comprise a pulse oximeter for measuring at least one of oxygen saturation and heart rate, and a microphone for recording at least one of respiratory and vocal sounds of the patient. The housing may, for example, include a pendant, and the pendant may include a finger-receiving surface (e.g., for the pulse-oximeter). As another example, the housing may include a patch. In some variations, the wearable device may further include a wireless communication system configured to transmit the one or more bodily metrics of the patient. Additionally or alternatively, the wearable device may include an alarm system configured to provide at least one of an audible notification and visual notification to remind the user to perform an assessment with the one or more sensors.

In some variations, a system for remotely managing a chronic respiratory condition of a patient includes a housing comprising a receptacle configured to removably receive a measurement device having one or more sensors for measuring one or more bodily metrics of the patient, a storage device configured to receive and store bodily metric data from the one or more sensors, and a wireless communication module configured to transmit the stored bodily metric data. In some variations, the measurement device may be a wearable device. In some variations, the system may include other components such as a microphone, a power supply to recharge a power source of the measurement device, and/or an alarm system configured to provide an audible and/or visual notification to remind the user to perform an assessment with the one or more sensors.

### BRIEF DESCRIPTION OF THE DRAWINGS

FIG. 1A is a schematic illustration depicting an exemplary platform for remotely managing a chronic medical condition of patients. FIGS. 1B and 1C are schematic illustrations depicting exemplary methods for remotely managing a chronic medical condition of a patient.

FIG. 2 is a schematic illustration depicting a measurement device for providing one or more bodily metrics of a patient for use in remotely managing a respiratory condition of a patient.

FIG. 3A is an exemplary variation of a measurement device including a wearable pendant. FIG. 3B is the device depicted in FIG. 3A, in use by a patient measuring bodily metrics.

FIG. 4A is another exemplary variation of a measurement device including a patch. FIG. 4B is another exemplary variation of a measurement device including an implantable device.

FIG. 5 is a schematic illustration of a user computing device for use in remotely managing a respiratory condition of a patient.

FIG. 6A is an exemplary GUI for facilitating entry of patient-reported bodily metrics. FIGS. 6B-6D summarize definitions corresponding to breathlessness, cough, and sputum scale (BCSS) scores as one form of patient-reported bodily metrics.

FIG. 7 is a schematic illustration depicting a docking station for use in remotely managing a respiratory condition of a patient.

FIGS. 8A and 8B are schematic illustrations depicting a docking station receiving a measurement device.

FIG. 9 is a schematic illustration depicting a predictive analysis system for use in remotely managing a respiratory condition of a patient.

FIG. 10 is a flowchart schematic of a method for remotely managing a respiratory condition of a patient.

FIGS. 11-17 depict exemplary GUIs of a web portal for a medical care provider for use in remotely managing a respiratory condition of patients.

FIG. 18 is an exemplary GUI of an updated patient status listing for a medical care provider for use in remotely managing conditions of patients.

FIG. 19 is an exemplary graph of a patient's BCSS scores and a trending index score for an upcoming respiratory event.

FIGS. 20A and 20B are exemplary graphs of a patient's SpO₂ and BCSS scores, respectively, over a monitoring period including a COPD exacerbation.

FIG. 21 is an exemplary graph of a patient's BCSS score over a monitoring period including multiple COPD exacerbations.

FIGS. 22A-22D are exemplary graphs of a patient's heart rate, breathing severity score, cough severity score, and sputum severity score, respectively, over a monitoring period.

FIGS. 23A and 23B are exemplary GUIs of a web portal for a medical care provider for use in remotely managing conditions of patients.

FIG. 24 is an exemplary summary statement for a medical care provider utilizing a platform for remotely managing patient medical conditions.

FIG. 25 is a schematic illustration of an exemplary variation for a method for managing a chronic medical condition of a patient.

### DETAILED DESCRIPTION

Non-limiting examples of various aspects and variations of the invention are described herein and illustrated in the accompanying drawings.

### Platform for Remotely Managing a Chronic Medical Condition

Various methods and systems for remotely managing (e.g., remotely monitoring) a chronic medical condition are described herein. For example, patient data may be obtained remotely through a remote condition management platform, and analyzed to assess patient condition (e.g., predict risk or likelihood of an upcoming clinical event, or otherwise characterize patient status). Patient assessments may trigger one or more alerts or action items (e.g., review of medical care data by a medical care provider and/or adjustments to a medical treatment plan, such as medication) that may, for example, reduce the need for more drastic measures such as emergency room visits, hospital admissions or re-admissions, ICU utilization, and/or days spend in the hospital. Accordingly, methods and systems such as those described herein may be used to remotely monitor patients and help prevent or reduce severity of adverse clinical events, thereby enabling better patient outcomes. In some variations, the platform may be used to remotely manage chronic respiratory conditions. For example, in some variations, patient data obtained remotely over a remote communication link may be used to predict the likelihood of an upcoming respiratory event for a patient having a chronic respiratory condition (e.g., COPD, asthma, sleep apnea, etc.). For example, the platform may be used to predict the likelihood of a COPD exacerbation (COPD flare) for a patient having COPD. However, the platform may additionally or alternatively be used to monitor any other suitable condition.

In some variations, the remote monitoring platform may be used by a suitable medical care provider (e.g., pulmonologist, other physician or nurse practitioner, other medical practitioner, suitable administrator associated with a medical practitioner, clinical staff or other auxiliary staff member or personnel, physician, or other qualified healthcare professional, etc.). In some variations, the medical care provider may include an auxiliary staff member under direct and/or general supervision of another medical care provider (e.g., supervising physician or qualified healthcare professional). For example, as described elsewhere herein, the auxiliary staff member may work at a different physical location (e.g., different building) and/or different time as the supervising medical care provider. Accordingly, in some variations, the remote monitoring platform may be used by a first medical care provider at a first location (e.g., at a virtual clinic) to remotely monitor or otherwise manage patients, while outsourced by a second medical care provider at a second location (e.g., at a physical clinic local to the monitored patient). For example, the remote monitoring platform may be used by a supervised healthcare professional or auxiliary staff member, who is in turn supervised by another qualified healthcare professional. As another example, the medical care provider may be providing designated medical care coverage (e.g., emergency or backup coverage) during a period of time in the absence of another (e.g., primary) medical care provider associated with one or more monitored patients. In some variations, the designated medical care coverage may be limited to a predetermined covering period of time (e.g., 72 hours). During such a period of time, the medical care provider may, for example, use the remote monitoring platform to monitor patients and respond to alerts (e.g., generated as described herein) appropriately in the absence of a primary medical care provider, such as by prescribing medication to treat an escalating medical condition.

### Patient Setup

Prior to being monitored, a patient is enrolled and integrated into the platform. For example, patient demographics (e.g., age, sex, height, weight, etc.), medical history (e.g., comorbidities, COPD exacerbation history, medication, etc.), a characterization of the severity of the patient's medical condition (e.g., a COPD patient's classification in the Global Initiative for Chronic Obstructive Lung Disease (GOLD) classification system), phone number and other patient contact information, contact information for medical care providers and/or designated care supporters, and/or any suitable patient information may be entered in the system and added to a patient file or account. This information may, for example, by provided through a web portal or the like and stored on a suitable server system.

In an exemplary variation, patient onboarding may be automated or semi-automated so as to be accomplished without significantly disrupting clinical workflow. Registration may, for example, be performed using a mobile computing device (e.g., tablet device, mobile telephone device, etc.) executing a software application. Medical staff at a doctor's office may, for example, provide the mobile computing device to a patient when the patient is being processed in a clinical setting (e.g., waiting in a doctor's office, awaiting discharge from a hospital, etc.). The software application executed on the mobile computing device may train the patient on remote patient monitoring by providing information regarding the remote patient monitoring services available (e.g., overview of how the service works, summary of benefits and advantages of the services, clinical data evidencing success, etc.). If the patient would like to opt in and sign up for remote patient monitoring, the patient can enter contact information (e.g., primary phone number and optional secondary phone number which may be used to uniquely identify the patient with the reported data, particularly in variations in which the patient reports data by calling into an automated phone system). The patient may further complete any necessary consent forms. In some variations, the software application may provide one or more field entries for receiving manually-entered contact information and/or consent (e.g., by typing information into a displayed user interface). Additionally or alternatively, the software application may be voice-enabled so as to receive and parse patient-recited contact information and/or consent. Such voice-enabled features may, for example, be helpful to elderly patients or other patients who may have difficulty typing or interacting with a graphical user interface. After receiving consent, the software application may generate an electronic patient agreement form (e.g., PDF file) including confirmation of consent (e.g., PDF file). After providing consent, the patient may then return the mobile computing device to medical staff, who may enter other patient information (e.g., patient name, medical record number, etc.) in the software application and confirm that the patient has been trained on remote patient monitoring. The doctor or other medical staff may then complete patient registration via software by completing doctor-side signatures and statements which are added to the patient agreement form and confirming submission of a prescribing order for remote patient monitoring. The doctor's prescribing order for remote patient monitoring may additionally be generated on the patient agreement form. The completed patient agreement form may, for example, be used for billing purposes and/or included in the patient's electronic health records.

Additionally or alternatively, some patients may be onboarded into a remote monitoring program by a program representative directly contacting each patient on a candidate patient list from medical care providers (e.g., clinics) such as by calling the patient directly, and/or contacting the patient over text, email or mail. For example, a patient may be contacted by a program representative through the patient's clinic (e.g., through the clinic's phone number, email messaging system, etc., with the permission of the clinic). The program representative (or a website accessible via a provided link) may provide an introduction or overview of the remote patient monitoring services available, explain that the patient has been identified as a candidate who may benefit from such services, request and receive patient consent over the phone for participating in the remote patient monitoring service, answer questions, and the like.

Additionally or alternatively, informational materials such as brochures or booklets may be distributed to candidate patients through the mail, or at the clinic office, with an invitation to return a form with patient consent to remote monitoring. Further completion of the onboarding or patient setup process may proceed following receiving patient consent. For example, patient supplies (e.g., monitoring equipment, instructions for providing patient assessments, etc.) may be provided to the patient at the patient's next clinic visit, may be delivered in-person at an at-home visit, or may be shipped, etc. Accordingly, training and other setup processes (e.g., consent) may be performed with more personal interaction.

As another example, the onboarding process may be initiated at least in part through an automatic messaging system (e.g., phone, email, etc.). For example, a patient may call a designated phone number to access an automated phone system that prompts the patient to dictate his or her personal and medical information (e.g., name, phone number, doctor, clinic, etc.). Furthermore, the automated phone system may prompt the patient to provide consent for enrollment with the monitoring platform, and this consent may be stored in a call log and recorded in the patient's medical record and proof of consent for remote monitoring. The information dictated by the patient may be transcribed using a suitable voice-to-text transcription service, and/or may be reviewed by an operator (e.g., at a clinic) to complete the onboarding process such as providing training, equipment, etc.

In some variations, the patient may be provided with suitable equipment for performing remote monitoring of bodily metrics, such as any one or more of the measurement device, user computing device, docking station, and/or other device variations described below. Furthermore, in some variations, patients may be given a laminated sheet or other document with a phone number to call on a repeated basis (e.g., daily, twice daily, hourly, etc.), with instructions to transmit or report at least one of the plurality of bodily metrics by calling the indicated phone number.

In some variations, upon being enrolled with the remote monitoring platform, a patient may be assigned to a platform operator (e.g., staff member) who may be a designated resource for troubleshooting issues associated with the patient. For example, as described below, transcription errors or other flags may occur during assessments when a patient provides his or her patient data over the phone. When such errors or flags occur, the issue may be forwarded directly to the platform operator assigned to that patient, in order for the platform operator to more efficiently address the resolve the issue (e.g., listen to a recording of the patient's voice and manually transcribe the patient data into text).

In some variations, the remote monitoring platform may be associated with a virtual clinic, and patient onboarding may include enrolling the patient with the virtual clinic. For example, the virtual clinic may be a professional corporation with a management services organization relationship with a technology provider. The virtual clinic may be staffed with one or more medical care practitioners (e.g., specialists or other practitioners) associated with the remote monitoring platform. The virtual clinic may provide remote monitoring for a single medical condition, or multiple different medical conditions. In some variations, a patient may be referred to a virtual clinic associated with the remote monitoring platform by the patient's local physician or other medical care practitioner.

In some variations, patients interested in remote monitoring through such a virtual clinic may indicate their interest through a virtual clinic-associated website (e.g., entering contact information through an enrollment form), emailing a suitable virtual clinic-associated email address, calling or texting a suitable virtual clinic-associated phone number, and/or other suitable business contact methods.

During patient onboarding, a patient may complete an intake questionnaire or other suitable intake form (e.g., dynamic or static questionnaire, such as a self-screening tool). The questionnaire may, for example, be tailored for a particular condition intended for remote monitoring. For example, a patient desiring to participate in remote monitoring for a chronic respiratory condition be asked to complete a questionnaire relating to personal respiratory medical history (e.g., diagnosis of any one or more of chronic lung diseases, use of an inhaler or nebulizer or other medications, use of supplemental oxygen, any recent emergency room or hospital visits, smoking history, etc.) or family medical history, and/or current symptoms of respiratory condition (e.g., coughing, breathing, sputum/mucus, etc.). Furthermore, the questionnaire may additionally or alternatively include generalized questions that are not necessarily medical condition-specific (e.g., relating to nature of the patient's social support network, medical contact information, etc.). The patient may also provide personal demographic information (e.g., name, insurance information and ID#, date of birth, address, etc.). In some variations, any of the foregoing information may additionally or alternatively be exchanged over email, etc.

Furthermore during patient onboarding with the virtual clinic, the patient may review and sign assignment of benefits and consent to copayment forms, as well as consent to one or more telehealth informed consent forms and/or other terms of use, privacy, etc. Consent may be provided through an online questionnaire or form, over the phone, and/or through mail, etc. A medical care provider (e.g., associated with the virtual clinic) may review the patient information provided through the questionnaire or other mechanisms, along with patient medication history and/or review of patient medical records. In some variations, the review of patient data may be performed in an asynchronous process (“store and forward”) where the patient doctor relationship is created without a live audio or video element (e.g., the patient's medical information may be transmitted from an originating site to the virtual clinic at a distant site, without the presence of the patient such as through live audio or video, and/or other real-time interaction). However, additionally or alternatively, in some variations the patient-doctor relationship may be established at least in part through a synchronous interaction involving a real-time interaction between the patient and the virtual clinic.

The medical care provider may use any one or more of multiple patient ID verification modalities prior to prescribing remote patient monitoring. In some variations, a text- and/or phone-based process may be used to verify patient identity. In some variations, patient identity may verified through a non-documentary identity verification process in which patient may provide personal identity information (e.g., name, date of birth, address, social security number or portion thereof, phone number, etc.) that is sent to an identity verification service that searches public and/or proprietary private databases for a match on the provided information. For example, a patient may be prompted to provide name, date of birth, address, and last four digits of his or her social security number to facilitate patient identity verification. As another example, a patient may be prompted to provide name and phone number to facilitate patient identity verification. However, any suitable combination of personal information may be provided for the purpose of patient identity verification. In some variations, non-documentary identity verification processes may make it easier for certain patient populations (e.g., elderly patients) to participate in the remote monitoring platform. However, additionally or alternatively, in some variations, a documentary identity verification process may be used, where an identity verification service may verify the authenticity of provided physical documents (e.g., driver's license or other registered identification document) and check against a picture of the patient in order to verify identity.

Based on the review of data, a medical care provider may establish a patient-doctor relationship and decide whether to prescribe remote patient monitoring to a candidate patient. If the patient is prescribed remote patient monitoring (e.g., using the remote monitoring platform as described herein), the medical care provider may create and prescribe a suitable care plan related to such remote monitoring. Throughout subsequent remote patient monitoring, the medical care provider may track patient data (e.g., bodily metrics including physiologic parameters as further described below) and adjust the remote monitoring plan as needed. Furthermore, in some variations the medical care provider may engage in interactive communication with the monitored patient (and/or a designated caregiver for the monitored patient), such as a phone call, an in-person visit, etc. For example, the medical care provider may interact with the monitored patient at least once per calendar month for at least a predetermined period of time (e.g., 20 minutes or more) or other suitable interval. The interaction between the medical care provider (e.g., clinical staff, physician, or other qualified healthcare professional) may, for example, include interactive communication over the automated phone system (e.g., by prompting a patient response by asking a question, requesting information, etc.).

### Patient Data

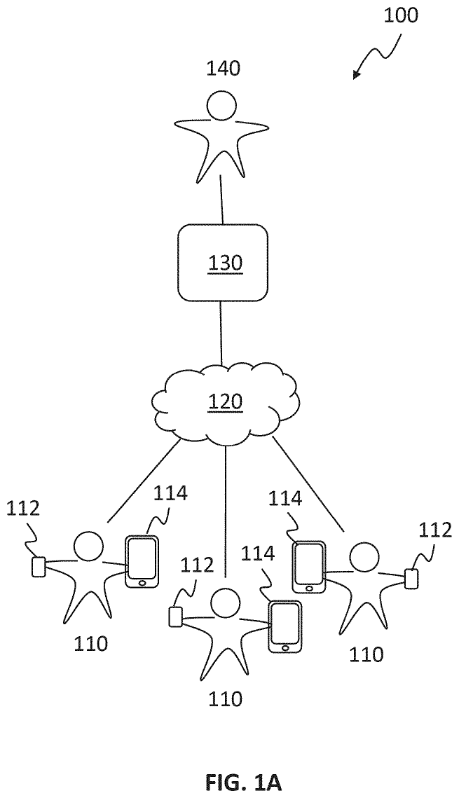

As shown in FIG. 1A, each of one or more patients 110 may interact with at least one measurement device 112 configured to obtain patient data (e.g., bodily metrics such as physiologic parameters for the patient) that may be analyzed to assess patient condition using the remote condition management platform 100. The measurement device 112 may include one or more suitable sensors configured to measure one or more bodily metrics. The type of one or more bodily metrics may depend on, for example, the type of medical condition being monitored. In some variations, multiple types of bodily metrics may be measured to facilitate remote monitoring of multiple medical conditions for a patient or multiple patients. In some variations, the measurement device 112 may be a wearable or handheld device, and/or may be removably received in a docking station. Exemplary variations of measurement devices 112 are described in further detail below.

The bodily metrics and/or other patient data may be provided over a network 120 to a predictive analysis system 130, for viewing and/or other use by a medical care provider 140 to monitor patient condition. As described in further detail below, the patient data may be provided through a reporting protocol performed by the patient as part of a regular (e.g., daily) assessment. For example, in some variations, at least some of the patient data may be provided by a patient through a user interface, such as dictated over the phone during a phone call with an automated phone system (or with an operator) with a programmable voice interaction.

As shown in FIG. 1B, an automated phone system associated with the predictive analysis system 130 may be configured to receive a phone call from a patient (150) on a regular basis (e.g., daily). The automated phone system may be configured with, for example, a programmable communications platform (e.g., programmable voice service, programmable text service, etc.). Any suitable programmable communications platform (e.g., communications platform as a service (CPaaS) with APIs) may be used to configure the automated phone system. In some variations, for example, the platforms provided by Twilio, Inc. or SignalWire may be used to configure the automated phone system as part of the remote monitoring platform. In other variations, the automated phone system may be configured as a relatively static platform (e.g., passively receives and records patient data without dynamic programmable aspects, etc.).

In some variations, the patient may be instructed to call the automated phone system and/or operator by a certain time every day (e.g., 10 AM), and failure to do so may prompt the automated phone system or other operator to separately contact the patient and/or medical care provider 140 for follow-up. For example, in some variations, an alarm may be triggered in the absence of an expected phone call (e.g., if the system has not received a phone call from the patient before a predetermined time for the day). The alarm may, for example include providing an SMS text message to the patient, a notification to the patient through a mobile application executed on a user computing device 114, an automated phone call to the patient (e.g., if the patient has not called into the system by a certain time, where the time deadline may be general to the system, or set specific to the patient, patient demographic, clinic, medical care practitioner, etc.), and/or an operator-performed phone call to the patient to provide a reminder for the patient to report patient data. Additionally or alternatively, the alarm may include a similar notification or phone call to a designated care supporter, such as a family member (e.g., spouse, sibling, son, daughter, etc.), a friend (e.g., neighbor or other contact), a member of a healthcare team, and/or a central service that can contact the patient and/or a designated care supporter(s) to check-in with the patient. Generally, the notification may indicate to the care supporter that the patient has not completed the expected (e.g., daily) assessment and asks or suggests that the care supporter follow-up with the patient to help ensure compliance with the reporting protocol.

In some variations the automated phone system may incorporate multiple escalating alarm levels. For example, a first alarm may be triggered in the absence of an expected phone call and may include the automated phone system or a live operator calling or otherwise reaching out in follow-up to the patient at a set time (e.g., customized to the patient). If the patient does not respond to this follow-up call, then this condition may trigger a second alarm that includes calling or otherwise contacting a designated care supporter(s).

FIG. 1B illustrates an exemplary variation of a method (101) of remotely monitoring a patient using the platform 100. Upon receiving a phone call (150), the identity of the caller may be confirmed as a particular patient (152). For example, the patient may be identified by identifying the phone number from which the phone call is originating, and matching the phone number to a patient (e.g., phone number associated with the patient such as during patient setup or onboarding).

In some variations, the automated phone system may provide a prerecorded outgoing voice message that prompts the patient or other caller to report patient data (162) or otherwise take certain actions. Patient data may be recited by the patient, entered on a keypad (e.g., touch-tone dial buttons, virtual representation of a numerical keypad, etc.), and/or the like. For example, the prerecorded voice message may prompt the patient with successive questions to directly elicit a submission of specific patient data (e.g., “What was your heart rate measurement today?”). The elicited patient data may include one or more bodily metrics such as biometric parameters, patient activity (e.g., inquiring about any recent visits to a medical care practitioner such as at a clinic, hospital, emergency room, etc.), or the like. The one or more questions to be asked may be generated (160) based on a predetermined list of questions. At least a portion of the questions asked to the patient may be static, or set to be asked by default, such as based on the medical condition being monitored. Additionally or alternatively, at least a portion of the questions asked to the patient may be asked at different frequencies (e.g., a question eliciting one or more biometric parameters may be asked every time the patient calls the automated phone system, while a question eliciting certain patient activity (e.g., “Have you been hospitalized or have you visited the emergency room in the past week?”) may be asked on a less frequent basis, such as weekly (or biweekly, or other suitable time period referenced in the question). Examples of questions for remotely monitoring a chronic respiratory condition, for example, are provided in more detail below.

Additionally or alternatively, at least a portion of the questions asked to the patient may be dynamic, or set to be asked in response to current patient data (e.g., patient answers to static questions, bodily metrics, etc.), such as in relation to alerts thresholds or other data analysis of the patient data. For example, in response to current patient data resulting in a patient being placed on an “alerts” or “watchlist” of patients (and considered more likely to require medical assistance), dynamic questions may be generated to gather further information to help characterize the nature of the medical condition. In other words, dynamic questions may include one or more follow-up questions that are customized to correlate to different risk level or alert thresholds. The types of dynamic questions may additionally or alternatively be medical condition-specific, practitioner-specific, clinic-specific, patient-specific, or otherwise customized.

In an illustrative implementation relating to remote monitoring of a respiratory condition, the patient may call into an automated phone system and report bodily metrics including heart rate, SpO₂, and BCSS score (and/or individual scores of breathlessness severity, cough severity, and sputum/mucus severity). However, in other examples, any of the above-described bodily metrics (e.g., blood pressure) may be measured with a suitable separate device (e.g., pulse oximeter) and reported over the telephone during an assessment call. Furthermore, the selection of bodily metrics for reporting may differ depending on stage of patient condition. For example, in some variations, the system may be specifically configured for some patients who are discharged from the hospital after a COPD exacerbation and may benefit from remote monitoring of a wider suite of bodily metrics. For example, a patient discharged from the hospital after a COPD exacerbation may additionally have his or her blood pressure remotely monitored (e.g., an automated phone system may prompt the patient to report his or her blood pressure during an assessment). Additional exemplary details relating to remote monitoring of a respiratory condition are described below, including analytical processes for characterizing patients on an “alerts” or “watchlist” of patients. In some variations, when a patient is placed on an “alerts” list based on currently reported patient data, dynamic questions may be generated to gather further information regarding the patient's respiratory condition. For example, the patient may be prompted to verbally describe generally how he or she is feeling. As another example, the patient may be prompted to describe the color of any sputum coughed up, whether there has been an increased volume of sputum, whether the patient is wheezing when exhaling, whether the patient is experiencing any sweats, fevers, chest tightness, shortness of breath, chest pain, etc., whether the patient has any increased ankle swelling, whether the patient's cough or breathing is interfering with sleep, whether the patient feels he or she can cope or manage with their condition that day, and any other suitable questions. The answers to these questions may be recorded and transcribed similar to that described below, and/or recorded and reviewed directly by a medical care practitioner.

The selection of bodily metrics for reporting may differ depending on the respiratory condition being monitored. As an illustrative example, in some variations, the system may be configured to monitor asthma patients. In these variations, the patient may, for example, be provided with a peak expiratory flow rate (PEFR) meter, and during the assessment (e.g., daily assessment) the patient may report PEFR value as measured by the PEFR meter, along with asthma-related symptoms. An example of asthma assessment is described in further detail below. As another illustrative example, in some variations, the system may be configured for some post-stroke recovery patients, who may benefit from remote monitoring of a wider suite of bodily metrics. For example, a patient recovering from stroke may additionally report blood pressure values (where variance in blood pressure may be correlated to increased risk of stroke recurrence) and other symptom scores relevant to stroke.

The dictated or otherwise entered patient data may be recorded (170), such as part of a raw data file stored in one or memory devices (e.g., a server). The patient data may be transcribed and/or parsed (172) using a suitable voice-to-text transcription model or service (e.g., Google Voice or the like). The collection of patient data (as well as during subsequent treatment of patient data, through transmission, analysis, etc.) may be performed in a HIPAA-compliant manner. For example, the remote patient monitoring platform may be designed with no “conduit exception”, such that storage of patient data is performed on one or more HIPAA-complaint servers, and any third party vendor having access to patient data (e.g., providing launch of phone calls, programmable voice services, voice-to-text transcription, etc.) has executed a suitable agreement (e.g., Business Associate Agreement) to operate in a HIPAA-compliant manner. Furthermore, in some variations (as further described below), some or all patient data may be de-identified to further ensure patient privacy. For example, in these variations, the de-identification may provide an additional layer of security in combination with HIPAA-compliant practices described herein, and/or a layer of security to prevent other third parties (e.g., those who do not sign suitable Business Associate Agreements) from having access to identifiable patient data.

In some variations, the transcription and/or parsing of recited patient data may undergo a verification process (174) to help ensure accurate data is being obtained through the telephone assessment. In the event of a transcription error or other flag, further review such as manual review or correction of patient data may be prompted (176). As one example in the verification process, a transcribed and/or parsed value of a bodily metric may be flagged for additional review in response to the transcribed and/or parsed value being outside of a predicted or expected range of values for that bodily metric. The predicted or expected range may, for example, be approximately centered around a historical average for that bodily metric for that patient (e.g., average of the values received over a certain number of preceding assessments, such as the most recent two, three, four, etc. assessments), or approximately centered around the value received for the most recent assessment. Such additional review may include, for example, a manual verification by a person to confirm or correct the transcribed and/or parsed value. For example, manual review may be prompted by sending a notification to an assigned operator who listens to the recording and addresses any issues with the transcription.

As another example, one or more errors in transcription of the patient's voice may be identified. One such error may, for example, be the result of low volume or low clarity of the caller's voice. In such instances, the automated phone system may prompt the caller to repeat himself or herself reporting the patient data (e.g., “Sorry, I didn't quite hear that. Please repeat”), up to a predetermined number of times before flagging the voice excerpt for further review (e.g., manual review as described above). Another error in the transcription of the patient's voice may be identified as the result of the voice-to-text transcription service failing to parse or transcribe the received words. Additionally or alternatively, a secondary transcription and/or parsing of the recited patient data may be performed by a different transcription or parsing service in order to confirm or correct the result from an initial service. As another example, a received audio recording of recited patient data may (e.g., as a matter of routine) be processed in parallel by multiple (e.g., two) automated audio-to-text transcription services. If all (or most) of the transcription services arrive at the same result, then the system may assume that the transcribed patient data is accurate. If the transcription services arrive at different results, then the recording of recited patient data may be flagged for additional review (e.g., manual review as described above). Accordingly, an automated phone system may include one or more of the above verification processes to account for any inaccuracies in transcribing and/or parsing patient data that has been recited over the telephone.

Once the patient data is satisfactorily transcribed and verified, the verified patient data may be stored (178) in a suitable HIPAA-compliant (i.e., compliant with the Health Insurance Portability and Accountability Act) data server system that is accessible for data analysis.

In some variations, the patient may recite a speech excerpt (e.g., sentence, paragraph, etc.), as described in further detail below, for recordation. The recordation may be processed, for example, to assess respiratory sounds, respiratory rate, vocal quality, and the like, as described below.

Additionally or alternatively, the automated phone system may provide one or more prerecorded outgoing informative messages. As an illustrative example, such a prerecorded outgoing message may include a reminder that the patient should call an emergency line (e.g., 911) or their doctor if they are experiencing a medical emergency. As yet another example, the automated phone system may provide one or more prerecorded outgoing messages associated with a certain day or time. As an illustrative example, a transmission confirmation message (e.g., “Thank you for providing your data today. Your data will be provided to your doctor's office shortly.”) may be played if the patient calls into the automated phone system during a predetermined time window for receiving patient assessments, such as typical business hours (e.g., Monday through Friday, between about 8:00 AM and 5:00 PM, etc.). As another illustrative example, a message informing the patient that there may be a delay in data transmission (e.g., “Thank you for providing your data today. Please note that your data will only be transmitted to your doctor's office during business hours Monday through Friday.”) may be played if the patient calls into the automated phone system outside of a predetermined time window for receiving patient assessments. Informing the patient that there may be a delay in patient data transmission may, for example, provide a disclaimer to help reduce medical liability for any delayed response to alerts, warnings, etc. that may result from a delayed receipt and/or viewing of the patient assessment data. It should be understood that any suitable combination of prerecorded outgoing messages may be provided (e.g., informing the patient that there may be a delay in data transmission due to being outside business hours in combination with instructing the patient to call an emergency help line if they are experiencing a medical emergency).

In some variations, the system may additionally or alternatively be configured to permit a patient to record a voice message. For example, the system may invite the patient to optionally leave a message by entering a submenu through dictation or other user input (e.g., the system may recite audio such as “Please don't forget to take your prescribed medication today. If you have any questions about your medication, please say or press ‘2’ . . . ”) upon which the patient may dictate a voice message. For example, during a telephone assessment, a patient may wish to record a voice message relating to his or her medical care (e.g., inquiring about or requesting an adjustment to an aspect of his or her treatment plan such as medication type or dosage, recording one or more questions or concerns about symptoms, etc.). The voice message may be transcribed into text, parsed into relevant action items (e.g., “check medication dosage”) such as with machine learning algorithms, and/or forwarded to the patient's medical care team as audio and/or text, in any suitable order. For example, the voice message may be accessible to a medical care practitioner viewing patient information through a web portal as described below, and may be played back to the medical care practitioner as text and/or audio. In some variations, the system may permit all patients to record a message with the system (e.g., automated system, operator, etc.). In other variations, the ability to record a message may depend upon the satisfaction of one or more conditions, such as a threshold trending index score (as further described below), a threshold relative patient ranking of the patient's trending index score, a threshold value for any one or more metrics (e.g., solely BCSS score), or the like. Furthermore, the system may additionally or alternatively permit a patient to request to be contacted directly by his or her medical care team. For example, the system may invite the patient to optionally request to be contacted by entering a submenu through dictation or other user input (e.g., the system may recite audio such as “If you would like to be contacted by your doctor, please press ‘3’ and we will notify your clinic to contact you . . . ”). Such a request may be forwarded to the clinic for follow-up action. Accordingly, in some variations, the remote patient monitoring system may encourage patient compliance with an overall medical treatment plan, by enabling a streamlined platform for communication between patients and their medical care team (e.g., providing reminders for the patient to take medication, allowing remote patient assessments and medication adjustments, simplifying the process for patients to contact their medical care team, etc.).

As described above, the voice of the patient may be transcribed (e.g., automatically by the automated voice system or manually by an operator) into text and parsed into relevant patient data (e.g., specific bodily metrics) and/or other information or messages from the user. In some variations, a patient may additionally or alternatively enter at least some patient data via a touchpad (e.g., keypad on a telephone). Accordingly, the automated phone system may function to gather patient data (e.g., bodily metrics) that are recited or otherwise provided over the phone.

Additionally or alternatively, the patient data may be transmitted over a remote communication link (e.g., over a wireless network 120) from one or more devices. For example, each of the one or more patients 110 may interact with a user computing device 114 configured to receive patient data (e.g., from the measurement device 112, entered directly from the patient, etc.), such as entering patient data through a user interface of a mobile application executed on the user computing device 114. The patient data may be provided directly from the measurement device, a docking station that receives the measurement device, and/or the user computing device. Patient data may be transmitted wirelessly, for example (e.g., through Bluetooth, WiFi, 3G/4G/5G cellular networks, etc.) and in a HIPAA-compliant manner.

To help ensure privacy and security of patient data transmitted to, from, and within the remote condition management platform 100, the platform 100 may incorporate various techniques. For example, FIG. 1C illustrates an exemplary method (102) similar to that shown in FIG. 1B, except that the caller identity may be masked or otherwise obscured (151) prior to being received and recorded by the automated voice system. For example, the phone call may be patched through a HIPAA-compliant masking storage server that masks the actual phone number with a proxy phone number or other stand-in identification code. The mapping between the actual phone number and the proxy phone number is not known to the programmable automated voice system, nor the voice-to-text transcription service. The caller's voice may additionally or alternatively be obscured with a voice filter that modulates the sound of the caller's voice. In variations in which the caller identify is masked (151), the patient may be re-identified (179) upon the patient data being stored in the HIPAA-compliant data storage server, by matching the proxy phone number to the actual patient phone number such as with a known, private key.

In some variations, a defined set of proxy phone number may be used in combination with timestamp information to mask a greater number of different patient identities. For example, a proxy number may be used multiple times to de-identify multiple patients over time. As an illustrative example, a proxy number may be used to mask a first patient at a first time (whereupon a first timestamp is also stored with the relationship between the first patient identify and the proxy number). Similarly, the same proxy number may be used to mask a second patient at a second time (whereupon a second timestamp is also stored with the relationship between the second patient and the proxy number). The first mapping between the first patient and the proxy number is thus distinguishable from the second mapping between the second patient and the proxy number, by virtue of the timestamps.

Furthermore, in some variations, patient data may be encrypted during transmission over the caller's phone to the automated voice system, encrypted within the automated voice system, encrypted during transmission from the automated voice system to a voice-to-text transcription service, encrypted within the voice-to-text transcription service, encrypted during transit to the data storage server, and/or encrypted within the data storage server itself. In some variations, patient data may be encrypted using asynchronous cryptography, as further described below.

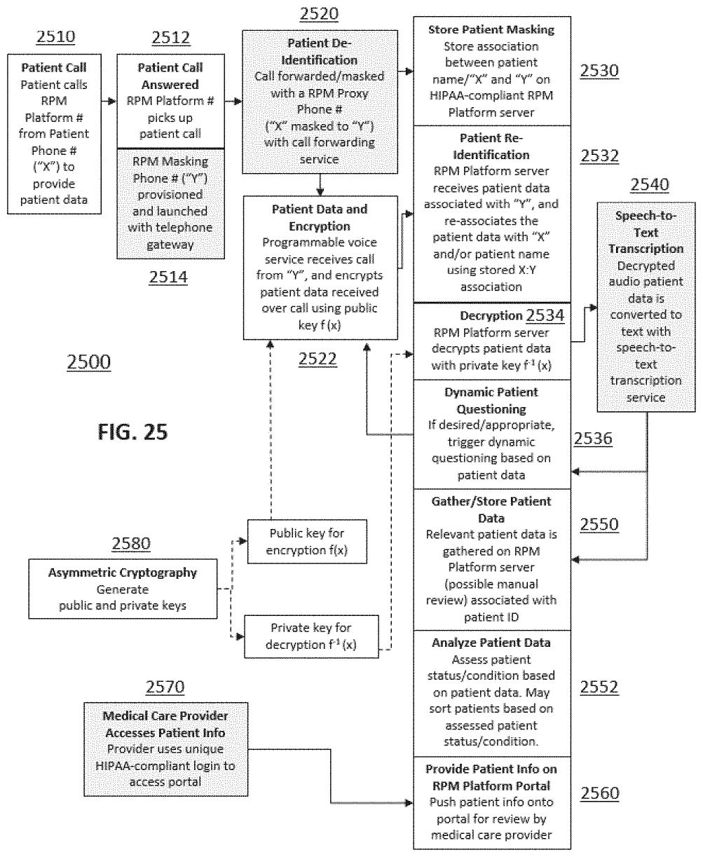

FIG. 25 illustrates in detail an exemplary variation of a method 2500 for remotely managing a medical condition for a patient, similar to that described above with respect to FIGS. 1B and 1C, where a patient may be remotely monitored using an automated phone system (e.g., a programmable voice service). In method 2500, cryptography may additionally be used to encrypt and decrypt patient data to further ensure patient privacy is maintained (e.g., comply with HIPAA). For example, as shown in FIG. 25, a provider of a remote monitoring service (or other suitable entity) may generate asymmetric cryptography keys (2580), including a public key to encrypt audio files recorded during a patient's phone call to the automated system, and a private key known only to other one or more entities for the purpose of decrypting audio files. These public and private keys may be used, for example, as described below.

As shown in FIG. 25, a patient may call a phone number associated with a remote monitoring platform (referred to in FIG. 25 as “RPM Platform”), using his or her personal phone number (2510), with the intent to provide bodily metrics or other patient data. The patient phone number “X” may already be identified as associated with the patient due to information provided during patient setup/enrollment or otherwise previously provided. The remote monitoring platform may then answer and launch the call (2512), such as with a telephone gateway that is HIPAA-compliant (e.g., under a business associate agreement). An example of a suitable telephone gateway is Amazon Connect. Furthermore, the telephone gateway may provision and launch a suitable masking phone number “Y” (2514).The patient call may be forwarded and masked using a call forwarding feature of the telephone gateway or other call forwarding service (2520). This enables the identity of the patient who called from his or her personal number “X” to be masked such that only the masking number “Y” is visible to the next entity receiving the call. The association or mapping between the patient identify (e.g., the patient number “X”) and the proxy number “Y” may be stored on a separate RPM platform server, such as an operator of the remote monitoring platform. The RPM platform server may be HIPAA-compliant.

The patient call, now masked with the proxy number “Y”, may be forwarded to a programmable voice service (2522) configured to prompt the patient to provide patient data such as bodily metrics, such as by asking questions or other prompts as described herein. The programmable voice service may, for example, initially ask the patient a set of one or more static questions. After each patient response, an audio file (or other file, such as including data created through the entering of patient data on a touch-tone dial keypad or other suitable keypad) is created and encrypted using the public key.

Each audio file (e.g., question response) may be individually sent (or sent in suitable batches) to the RPM platform server. When the audio file for a question response is received by the RPM platform server, the audio file is associated with the proxy number “Y”, and the RPM platform server may re-associate the patient data with the original patient identity using the stored mapping between “X” and “Y” (2532). Furthermore, the RPM platform server may decrypt the encrypted audio file using the private key dedicated for such purpose (2534). With the audio file both decrypted and associated with the real patient name and number, the audio file may be sent to a suitable speech-to-text transcription service (2540), which converts the audio file into text. The speech-to-text transcription service may also be HIPAA-compliant under a suitable business associate agreement or the like.

If there is a transcription error, then manual review may be performed to determine next steps as described above. If there is no transcription error, then the text transcription may be sent back to the RPM platform server, and the patient responses (patient data) may be tabulated to determine whether additional or follow-up dynamic questions may be necessary (2536). For example, as described elsewhere, specific values for certain bodily metrics may be compared to one or more predetermined thresholds that trigger dynamic questioning (the thresholds may be customizable for each patient, for a clinic, for a medical care practitioner, etc.). If dynamic questions are triggered, then the programmable voice service asks additional questions to the patient, and the patient responses (as additional patient data) may be encrypted, decrypted, transcribed, etc. again. This dynamic questioning cycle may be repeated as many times as necessary based on the thresholds and/or other triggering rules. Once all questions are asked and patient response stored on the RPM platform server, any of the encrypted data that was transiently stored on the programmable voice system may be deleted.

Relevant patient data may then be gathered and stored on the RPM platform server (or other suitable server). For example, the audio file and/or text transcription may be stored (2550) associated with the actual patient name and/or his or her personal phone number “X”. If any values fall out of a predicted range (or other flag is triggered), or if a transcription error persists, then this data may be held on the server for further manual review. For example, a staff person associated with the remote monitoring platform may listen to the audio file to confirm, correct, or otherwise clarify the system's understanding of the contents of the audio file. The patient data may then be analyzed (2552) and/or provided on a suitable portal or other user interface (2560) for communication to a medical care provider for assessment, as described in further detail herein. For example, a medical care provider may utilize HIPAA-compliant login credentials to access a portal providing patient data (2570), such as suitable web interface, computing application, etc.

### Data Analysis and Alerts

As shown in FIG. 1B, the patient data may be used to assess the patient (180), and a medical care provider may be allowed to view patient data and/or patient assessments (190). For example, the predictive analysis system may perform analytics to assess the received patient data and may transmit or otherwise communicate an alert (e.g., a programmed alert) to at least one medical care provider (e.g., pulmonologist, other clinical staff or auxiliary staff member or personnel, physician, or other qualified healthcare professional, etc.). As described above, in some variations, the medical care provider may include an auxiliary staff member under direct and/or general supervision of another medical care provider (e.g., supervising physician or qualified healthcare professional). For example, as described elsewhere herein, the auxiliary staff member may work at a different physical location (e.g., building) and/or different time as the supervising medical care provider. Accordingly, in some variations, the remote monitoring platform may be used by a first medical care provider at a first location (e.g., at a virtual clinic) to remotely monitor or otherwise manage patients, while outsourced by a second medical care provider at a second location (e.g., at a physical clinic local to the monitored patient). For example, the remote monitoring platform may be used by a supervised healthcare professional or auxiliary staff member, who is in turn supervised by another qualified healthcare professional.

For example, the patient data may be analyzed via machine learning and/or other artificial intelligence techniques, and an output may be created as an assessment of patient risk (e.g., severity of patient condition, or predicted likelihood of a respiratory or other clinical event, such as a COPD exacerbation). For example, the patient data across multiple patients may be analyzed in order to characterize each patient's risk of experiencing a respiratory or other clinical event. Multiple monitored patients may, for example, be ranked in accordance to their assessed risk of experiencing a respiratory or other clinical event, such that higher-risk patients (e.g., patients with a higher determined risk of experiencing a respiratory or other clinical event) may be identified. For example, patients may be tiered into an “alerts” list of patients and a “watchlist” of patients, where the patients on the alerts list may be considered more likely to require medical attention than those on the watchlist, while patients on the watchlist may be considered more likely to require medical attention than those on neither the alerts list nor the watchlist.

The medical care provider may then follow up with the identified higher-risk patients, such as to determine whether further medical intervention is appropriate. For example, an alert based on the assessed patient risk may be transmitted or otherwise communicated to a medical care provider. The alert may be communicated, for example, if the assessed level of risk for a patient (or ranked risk level, etc.) satisfies one or more predetermined thresholds. Such predetermined thresholds may be set by the medical care provider and/or be default suggested settings. In some variations, the rules associated with triggering alerts may be specific to the medical care provider 140, specific to one or more patient demographics or characteristics (e.g., patients with N COPD exacerbations in a preceding time period such as the previous month, six months, or twelve months), specific to patient baseline or historical trends, environmental or seasonal considerations (e.g., poor air quality due to fires or pollen, etc.), and/or other suitable considerations.

Furthermore, a medical care provider may select suitable alert conditions, such as thresholds and/or other conditions for triggering an alert notification associated with a high-risk patient (e.g., a patient with a relatively high likelihood of experiencing an upcoming clinical event.) The alert conditions may be patient-specific (e.g., custom or dependent upon patient characteristics as a whole), demographic-specific (e.g., dependent upon GOLD classification), or default settings. Similarly, a medical care provider may additionally or alternatively select preferred factors for a trending index (as further described below), such as indicating whether to use absolute changes or relative changes in bodily metrics or other settings. For example, a medical care provider may select settings to enable monitoring of a patient based on the patient's changes relative to other patients (e.g., through ranking), and/or monitoring of a patient based on the patient's historical trends (e.g., relative to past bodily metrics).

Upon receiving the alert, the medical care provider may review medical records and/or follow-up in a suitable manner with the patient (e.g., through an online web or software portal). The medical care provider may choose to review the patient data and any other relevant information in the patient's medical records, and may modify a medical treatment or other management plan (e.g., adjust medication) for the patient accordingly. Such modification may be performed remotely or in person. Further review and assessment of the patient data and/or additional modifications to the medical treatment may be subsequently performed, such as during a monitoring period. For example, members of a healthcare team may continue to monitor the patient using the system after altering the management treatment plan (e.g., to assess how the patient responds). If needed, the system may allow the healthcare team to contact the patient (e.g., through video chat, phone, text message, other messaging, etc.) and/or allow the patient to request such communication with the healthcare team and/or review his or her own patient data.

### Practitioner Reporting

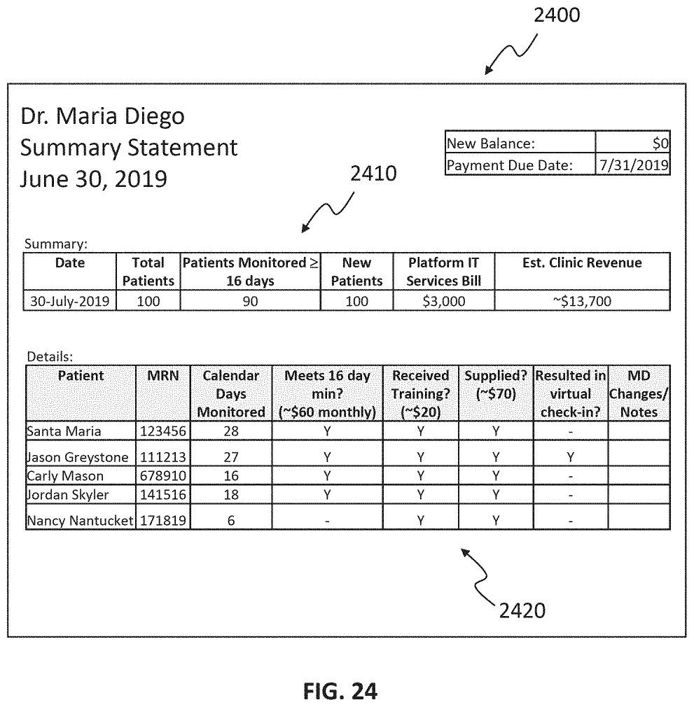

In some variations, a user interface may provide a practitioner or clinic summary relating to monitored patients, and/or an exported summary statement thereof. For example, as shown in FIG. 24, a summary statement 2400 for a medical care provider may provide a financial and care overview of patients being remotely monitored with the remote condition management platform (e.g., for remote monitoring of a respiratory condition, or other medical condition). The summary statement 2400 may include, for example, a financial overview 2410 including total number of patients monitored, number of patients satisfying a minimum number of days of monitoring required for reimbursement (e.g., number of patients for which bodily metrics have been received each day for at least a minimum number of calendar days in a calendar month, such as 16 days), number of new patients, platform administrative costs, and overall estimated clinic revenue resulting from the monitoring of patients. The summary statement 2400 may also include a detailed patient overview 2420 providing a breakdown of extent of monitoring and other services provided to each patient. Some or all of the information on the summary statement may be formatted for easy and efficient forwarding to a third party medical billing company, such as for reimbursement purposes.

### Patient Engagement

Consistent patient engagement may improve the accuracy and effectiveness of remote patient monitoring service. However, some patients may tend to be discouraged from consistently engaging with the remote monitoring service (e.g., because such monitoring services may be a frequent reminder of their illness or health condition). Accordingly, in some variations, methods for remotely managing patient conditions may benefit from including incentives that improve patient engagement and morale. For example, as part of patient onboarding (or as a follow-up to getting the patient set up with the remote monitoring service), a welcome kit may be provided to the patient. In addition to monitoring equipment (e.g., pulse oximeter) and description of the service, the welcome kit may include a welcoming and encouraging message, a reward such as a gift card, etc. in order to thank the patient for joining the community engaging with the remote patient monitoring service. As another example, a reward may be provided to the monitored patient periodically (e.g., every month, every other month, etc.) or in response to milestones (e.g., a continuous streak of successful daily assessments provided, a threshold number of days (such as 16 days) in a month for which assessments have been provided, etc.). The reward may include, for example, a gift card, entry into a raffle, promotional gifts, recognition in a community or clinic newsletter, etc.

In other examples to help encourage patient engagement (for example, among patients who may be elderly), pleasant or reminiscent content may be provided to patients who successfully complete daily assessments. For example, methods may include providing content (e.g., music clips, journalistic content such as selected content from newspapers or magazines, etc.) that is associated with patients' younger days. In some variations, methods for encouraging patient engagement with a remote monitoring service may be patient-tailored. For example, after identifying a patient characteristic (e.g., age, hometown, home country, etc.), reminiscent content associated with that patient characteristic may be provided to the patient upon successful completion of a daily assessment, continuous streak of daily assessments, threshold number or proportion of daily assessments, etc.

In some variations, the level of patient engagement may be tracked among a population of monitored patients. For example, patients who successfully complete a threshold number or proportion of daily assessments may be identified as in compliance with the remote monitoring program. In some variations, the patients who successfully complete at least 16 daily assessments in a month (e.g. or other threshold number or proportion as required for insurance reimbursement) may be identified. A medical care practitioner (or clinic) may receive a list or statement of the identified patients who are in compliance (along with other useful information such as exact number of days data was transmitted, appropriate insurance reimbursement codes, etc.), which may in turn simplify and streamline the billing process for insurance reimbursement (and/or for helping to assess success or usefulness of the remote monitoring program). For example, in some variations, the web portal for a medical care provider (as described above) may include a “billing” tab for tracking patient participation (e.g., the number of days patient data is transmitted per patient per calendar month or other suitable time period). Additionally or alternatively, a report summarizing patient participation may be provided periodically (e.g., monthly) to the medical care provider.

Patient engagement and overall compliance with providing assessments may be further encouraged through other processes. For example, in some variations, a patient may earn rewards for completing assessments. For example, if the patent performs the full assessment to generate patient data (e.g., each successful completion of an assessment, or a consecutive sequence of multiple successful completions of an assessment), then a reward can be given to the patient (or suitable beneficiary such as a designated care supporter, or group of choice). Rewards may include, for example, points, tokens, money, cryptocurrency, and/or other items of value, etc. that may be redeemed for gift cards or other rewards. In some variations, rewards may include a donation to a group of choice such as a charity or foundation, which may be encourage patients to continue completing assessments, knowing that their compliance with remote monitoring may help others. For example, a reward for compliance with remote monitoring may include a donation to medical research, such as research related to the patient's monitored condition, which may incentivize patients to comply knowing that their actions will help themselves as well as others with similar conditions.

Other behavioral tendencies of patients may be leveraged to increase compliance. For example, a patient and/or care supporter(s) may periodically (e.g., weekly, monthly, etc.) receive a report summarizing their compliance with completing assessments, where compliance may be tracked relative to the patient's historical record (e.g., so the patient may strive to improve compliance over time) and/or other patients' compliance in completing their assessments. Such a periodic report may further include patient health data (e.g., summary of trends in their bodily metrics over time, etc.). As another example, in some variations, the user experience when the patient is completing or has completed assessments may be enhanced with positive feedback such as complimentary or congratulatory statements or feel-good sounds (e.g., verbal encouragement such as “Thank you for taking care of yourself”, pleasant sound effects indicating approval, etc.). In these variations, the positive feedback may be varied (e.g., different statements or sounds each day) such that patients may look forward to completing assessments to hear the newest feedback. For example, the patient may be rewarded with a message that is provided to the patient during their call to the automated phone system (e.g. “Congratulations, you have provided your data 4 days in a row. Keep up the good work!”).

Additional various aspects of exemplary systems and methods using the platform 100 are described in further detail below.

### Systems for Remotely Managing a Chronic Respiratory Condition

In some variations, the remote condition management platform may be used to manage a chronic respiratory condition. Generally, in some variations, a system for remotely managing a chronic respiratory condition may include a single wearable device, single non-wearable device, or a combination of multiple wearable and/or non-wearable devices. For example, the system may include a wearable measurement device including one or more sensors for measuring one or more bodily metrics of the patient. The system may further include a docking station configured to removably receive the wearable measurement device. The wearable measurement device and/or the docking station may include a wireless communication system configured to transmit the bodily metrics (e.g., to a server). Additionally or alternatively, the system may include a user computing device configured to receive user input of one or more bodily metrics (e.g., through a mobile application), and the user computing device may include a wireless communication system configured to transmit bodily metrics. The bodily metrics may be analyzed by a predictive analysis system for predicting likelihood of a respiratory or other clinical event based at least in part on the bodily metrics.

### Measurement Device

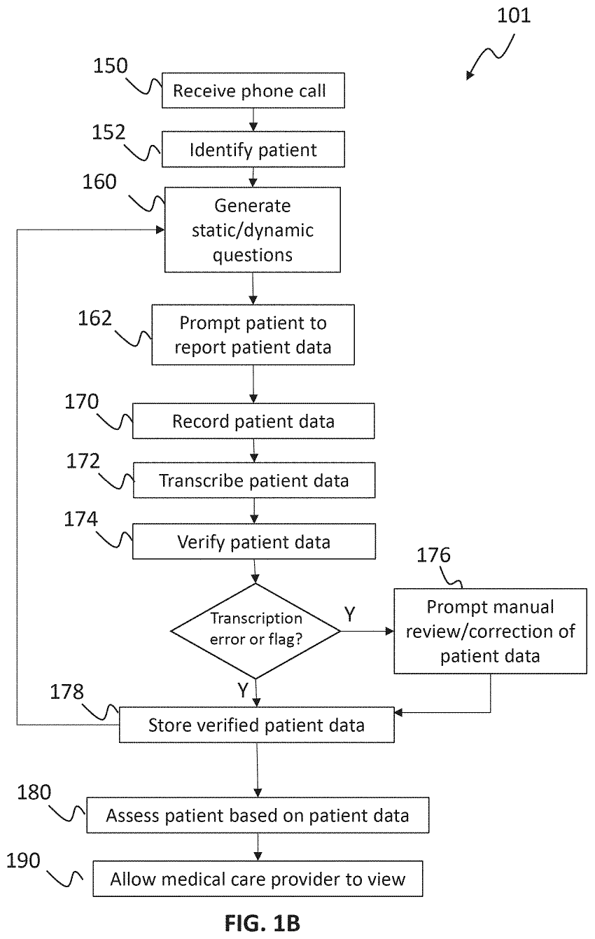

The measurement device may be wearable and include a housing and one or more sensors for measuring one or more bodily metrics of the patient. The one or more sensors may include, for example, a pulse oximeter configured to measure oxygen saturation and/or heart rate (and/or other cardiac characteristics) of the patient, though other sensors may additionally or alternatively be included in the measurement device, as described below. In some variations, the measurement device may further include a microphone configured to record sounds (e.g., respiratory sounds such as breathing and/or coughing, vocal sounds such as speech, etc.). For example, as shown in FIG. 2, an exemplary variation of a measurement device 200 may include at least one sensor 210, at least one microphone 220, and various electronic devices such as at least one processor 230, at least one memory device 240, and at least one network communication interface 250. Other components, such as a power supply, may further be included in the measurement device. In some variations, the system for remotely managing a chronic respiratory condition may include multiple measurement devices, such as a combination of multiple variations described below.

### Housing and Form Factors

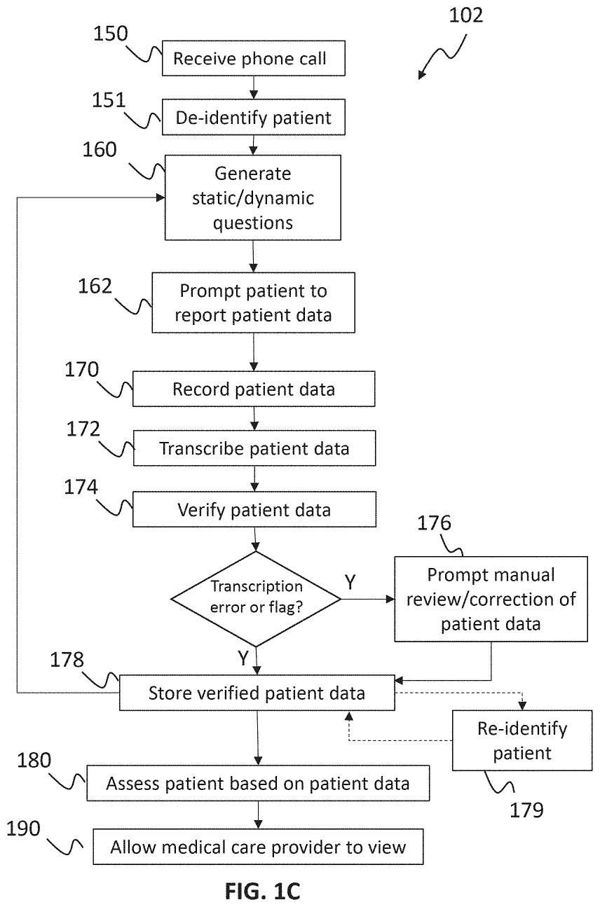

The measurement device may include a housing configured to provide a structure or other substrate for supporting the various other components of the measurement device such as sensors, computing devices, etc. The housing and measurement device may take any suitable shape or form. For example, as shown in FIGS. 3A and 3B, in some variations, the housing may include a pendant 300 that can be worn by the patient. The pendant 300 may be coupled to an attachment feature such as a chain, thread, or other elongated member that can be draped around the neck of the patient as shown in FIG. 3A. In other similar variations, the housing may be similar to pendant 300, but may additionally or alternatively include another suitable attachment feature such as a ring or partial ringed member, which may be rigid or semi-rigid (e.g., such that the measurement device may be worn around the neck like a choker necklace, or around an arm, wrist, ankle, leg, or finger, etc.), clip, pin, and the like. Furthermore, in some variations, the housing may be similar to pendant 300 may omit an attachment feature, such that the measurement device may be a handheld device. The housing may be waterproof or water resistant, such that the patient may wear or hold the measurement device during daily activities such as showering, bathing, exercising, sleeping, etc. with lowered risk of damaging the measurement device and its components.

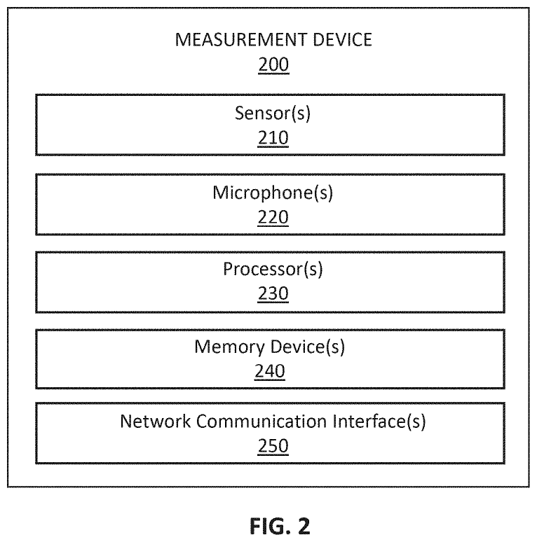

As another example, as shown in FIG. 4A, the housing may include a patch 400. The patch 400 may have one or more sensors (described in further detail below) layered or embedded in the patch. The patch 400 may be applied to a target skin area of the patient, such as the chest or back, and attached via an adhesive backing, straps, ties, and/or any suitable attachment feature. In some variations, at least portion of the patch 400 may be flexible and conform to the body of the patient. In some variations, the patch 400 may be configured to be worn substantially continuously for a predetermined period of time such as between about 24 and about 48 hours, between about 3 days and 5 days, between about 5 days and about 7 days, between about 1 day and about 7 days, between about 1 week and about 2 weeks, between about 1 week and 4 weeks, between about 1 month and 2 months, between about 1 month and 3 months, or more than 3 months. The patch 400 may be removed and replaced by a new or refurbished patch. Like the housing described above, the patch may be waterproof or water resistant. In other similar variations, the housing may include a cuff (e.g., blood pressure cuff), wristband, garment, and the like.

Furthermore, while in some variations the measurement device is a wearable device as described above, it should be understood that in other variations, the measurement device may be configured as a non-wearable device. One example of a non-wearable measurement device for providing one or more bodily metrics is an external monitor such as a bedside monitor or home virtual assistant device (e.g., similar to Amazon Echo® or Google Home™ devices), a set top box service (e.g., similar to Apple TV®), or other smart appliances such as a clock, radio, and the like.

The external monitor may be similar in many ways to the docking station described below. In some variations, for example, the monitor may include a speaker and ask the patient questions (e.g., questions regarding any changes in the patient's breathing capabilities, sputum/mucus, cough, etc.). Like the other variations of the measurement device described herein, the monitor may include an alarm reminder to notify the patient to take medications and/or complete his or her daily assessment of bodily metrics. The monitor may further include one or more screens to allow video conferencing between the patient and the healthcare team.

Another example of a non-wearable measurement device is an implantable device. An implantable measurement device may, for example, be placed subcutaneously in a patient. For example, as shown in FIG. 4B, an implantable measurement device 410 may be placed under the skin near the patient's suprasternal notch. The device sensors may collect bodily metrics (as further described below) at regular intervals (e.g., at least once daily), and collection of bodily metrics by the device sensors may be automatic (e.g., not in response to an alarm trigger). However, in some variations, at least some other bodily metrics may be collected in response to an alarm trigger (e.g., user-entered metrics). In some variations, the implantable measurement device may include a power supply (e.g., battery) with a lifespan of at least 6 months, at least 1 year, at least 2 years, or more. The power supply may be recharged wirelessly (e.g., RF, inductive coupling, etc.), such as through an external bedside charger or handheld charger.

In some variations, as shown in FIG. 3B, the housing may include at least one patient-contacting surface that enables measurement and/or recordation of one or more patient bodily metrics by various sensors as further described below. For example, the housing may include a surface configured to receive a finger for measuring oxygenation saturation of the patient. Additionally or alternatively, the housing may include a surface that may be arranged over the suprasternal notch for a microphone to record respiratory and/or vocal sounds. Other sensors may passively collect bodily metrics or other patient data, and/or may collect data upon initiation by the patient (e.g., through detection of skin contact such as with a capacitive sensors, button, etc.). For example, in some variations, recording of breath or vocal sounds may be triggered when the patient places his or her finger in a pulse oximeter (or other finger-receiving component) portion of the measurement device, and/or when the measurement device is placed over the suprasternal notch.

### Alarm

In some variations, the measurement device (and/or user computing device described below) may include an alarm trigger configured to activate at one or more predetermined times or intervals (e.g., daily) so as to remind a patient to perform an assessment. In some variations, the alarm can be configured to activate at approximately the same time a patient is indicated to take his or her medications (e.g., daily inhaler for COPD). However, the alarm can additionally or alternatively be configured to activate at any convenient time, such as when the patient routinely wakes up in the morning (e.g., 7 am) or goes to bed (e.g., 10 pm). The alarm may include audible notifications (e.g., sound such as musical tone, beep, etc.), visual notifications (e.g., flashing lights), physical notifications (e.g., vibration through a vibratory motor), or any combination thereof. Settings such as time, frequency, etc. for the alarm trigger may be modified by a healthcare provider (e.g., through a web portal or other user interface), by the patient (e.g., through a user computing device such as a device with a mobile application specific to the system), and/or directly on the measurement device.

Additionally, in some variations the user computing device may additionally or alternatively include an alarm. For example, in variations in which the measurement device is implantable and gathering certain bodily metrics (e.g., oxygen saturation, heart rate, respiratory rate, etc. as described herein) without any external prompt, the user computing device may be configured (e.g., through a mobile application) to notify the patient to enter user-entered bodily metrics (e.g., breathlessness, cough, and sputum scale (BCSS) scores) as described below.

The alarm may continuously or intermittently continue until a patient performs or begins to perform the actions required by the system (e.g., perform assessment of bodily metrics). For example, as part of an assessment of bodily metrics, patient may need to measure oxygen saturation, heart rate, and report BCSS scores. If the patient begins the assessment, the alarm may be paused or terminated. For example, the device may detect that the patient has begun the assessment by detecting that the patient has placed his or her finger on or in a pulse oximeter in the device. If the patient does not begin the assessment, or only a portion of the assessment is performed (e.g., if the patient fails to enter BCSS scores after a predetermined time after obtaining data from the pulse oximeter), then the alarm may resume.

In some variations, if the patient fails to respond to an alarm, then a notification may be communicated to a designated care supporter. For example, the designated care supporter may be a family member (e.g., spouse, sibling, son, daughter, etc.), a member of a healthcare team, and/or a central service that can contact the patient and/or designated care supporter(s) to check-in with the patient. The notification may, for example, be in the form a text message, phone call, notification through a mobile application and the like. Generally, the notification may indicate to the care supporter that the patient has not completed the expected (e.g., daily) assessment and asks or suggests that the care support follow-up with the patient to help ensure compliance. Additionally or alternatively, compliance may be further encouraged through other processes such as rewards for completing assessments, or leveraging other behavioral tendencies, as described above with respect to the remote condition management platform 100.

### Sensors

The measurement device may include one or more sensors configured to measure one or more bodily metrics. Objective, measurable bodily metrics generally may include, but are not limited to, oxygen saturation, carbon dioxide saturation, pH, respiratory rate, respiratory sounds, vocal sounds (e.g., speaking quality), cough frequency or quality, accelerometer with step counting and/or positional data, impedance, resistance, impulse oscillometry, temperature, volatile organic compounds in breath, exhaled condensate analysis, air quality sensor, weight, blood pressure, heart rate, heart rate variability, supplemental oxygen level, estimations of central venous pressure, peak expiratory flow, photo/audio/video of the patient or sputum/mucus, and the like. More detailed example as described below.

Oxygen saturation. In some variations, the measurement device may be configured to measure oxygen saturation of the patient. The level of oxygen in COPD patients may be indicative of severity of the disease condition. Oxygen level may be measured as blood oxygen level (PaO₂), or as saturation of peripheral oxygen (SpO₂). Normal SpO₂ in healthy individuals may generally range above 95%, while patients with COPD tend to have SpO₂ typically between about 88% and 92%. Changes in SpO₂ may signal an impending or current COPD flare, for example. Oxygen saturation may be measured by, for example, a pulse oximeter in the measurement device. By analyzing oxygen saturation alone or in combination with one or more bodily metrics (e.g., as described herein), the system may predict respiratory events such as COPD flares.

Heart rate. The measurement device may be configured to measure heart rate, or heart rate variability. These measurements may be performed by, for example, a pulse oximeter in the measurement device. Additionally or alternatively, the heart rate may be manually obtained by the patient and entered through a suitable user interface such as on the user computing device (e.g., through a mobile application).

Carbon dioxide saturation. In some variations, the measurement device may be configured to measure carbon dioxide saturation. Patients with COPD often retain carbon dioxide (CO₂) due to numerous reasons related to their condition, including ventilation/perfusion mismatch, haldane effect, and others. Patients with COPD are at risk of hypercapnia and sequalae, which can be worsened with too much supplemental oxygen. Thus, there may be a desire to keep patients at a SpO₂ target between about 88% and about 92%. Remotely monitoring the levels of oxygen saturation, CO₂ saturation, and/or supplemental oxygen levels in aggregate may improve management of COPD patients.

Typically, gas levels in COPD patients during acute hospitalization often involve measurement of arterial blood gases (ABGs), which necessitates an invasive approach of drawing blood from the patient. ABG analysis may include pH (acidity/alkalinity), paO₂ (partial pressure oxygen), paCO₂ (partial pressure carbon dioxide), and/or HCO₃ (bicarbonate). Changes and trends in these values are informative regarding the status of a patient with COPD, for example, if a patient is adequately compensating for an acid/base disturbance. Accordingly, in some variations, the measurement device may be configured to monitor (e.g., non-invasively) one or more bodily metrics from which one or more of the ABG assessments may be made.

For example, the measurement device may be configured to non-invasively monitor exhaled breath and measure end-tidal CO₂ pressure (PetCO₂), which may be correlated to PaCO₂, a major indicator for assessing a COPD patient. Because PetCO₂ alone might not always be a reliable estimate of the PaCO2 for COPD patients for physiological reasons (and because the gas needs to be sampled directly at the patient's airways which can be difficult to accomplish), one or more other bodily metrics may be monitored in combination with PetCO2 to remotely manage patients with COPD.

As another example, the measurement device may additionally or alternatively be configured to perform transcutaneous monitoring (PtcCO₂), which may be correlated to PaCO₂. For example, the measurement device may include one or more sensors at the skin surface (e.g., for application to the chest, back, etc. that detects CO₂ gas diffused through body tissue and skin. The sensor may be warmed, thereby inducing a local hyperemia, which increases the supply of arterial blood to the dermal capillary bed below the sensor. In general, this value correlates well with the corresponding PaCO₂ value.

In contrast to conventional PtcCO₂ monitors, in some variations the measurement device as referred to herein may be configured to provide electrical stimulation (e.g., via surface electrodes, needle electrodes, etc.) to increase local blood flow to the skin. Such electrical stimulation may, in some variations, be combined with a safe amount of heat to induce a suitable increase in local blood flow to the skin. Advantageously, such electrical stimulation may increase local blood flow without excessively raising skin temperature, in contrast to conventional PtcCO₂ monitors which increase skin temperature (for example, to about 42° C.) which can be uncomfortable to patients and/or lead to burns on the skin if placed for too long. In some variations, the PtcCO₂ monitor may be housed in a cuff (e.g., similar to a blood pressure cuff), or other suitable housing structures as described in further detail above.

Although correlatable to PaCO₂, the measured transcutaneous value PtcCO2 tends to be higher than the arterial value PaCO₂. The shift of PtcCO₂ towards higher values may be attributable to two main factors. First, the elevated temperature may increase local blood and tissue PCO₂ by approximately 4.5%/° C. (anaerobic factor). Second, the living epidermal cells produce CO₂, which contributes to the capillary CO2 level by a constant amount (metabolic constant). For example, skin metabolism may increase the PtcCO2 by approximately 5 mm Hg. Accordingly, in some variations, a suitable correction factor may be applied to the transcutaneous value PtcCO2 during analysis, in order to provide a reading that corresponds as close as possible to PaCO2 standard arterial value.

In another variation, a sodium bicarbonate breath test (SBT) may be used to remotely estimate arterial paCO₂ as a bodily metric. The SBT involves delivery of a known quantity of a ¹³C-labeled substrate (sodium [¹³C]bicarbonate) into the patient such as through ingestion, inhalation, injection, etc. The substrate is metabolized such that the ¹³C tracer is released and circulated as ¹³CO₂ in the patient's body and eventually excreted in the patient's breath, such that a pattern of ¹³CO₂ enrichment in the patient's breath may be identified. Accordingly, at one or more known time intervals, the patient may exhale into the measurement device which collects air and can measure the amount of exhaled ¹³CO₂, from which arterial paCO₂ can be approximated. Results of SBT are transmitted remotely along with any other bodily metrics as described herein, and changes in the SBT results may be considered as part of the risk assessment for prediction of a respiratory event, such as a COPD exacerbation.

pH. In some variations, the measurement device may be configured to measure pH levels or changes thereof over time. For example, the device may include a non-pulsatile near infrared spectroscopy device, which has the ability to simultaneously determine muscle oxygen saturation and muscle pH. A measurement of pH may be helpful in monitoring a respiratory condition, because elevated CO₂ levels (e.g., as experienced by patients during a COPD exacerbation) are countered by an increase in physiologic bicarbonate (respiratory acidosis with compensatory alkalosis). Accordingly, understanding any changes in pH levels can be informative regarding a respiratory condition such as COPD.

Exhaled condensate analysis. Markers of airway inflammation and oxidative stress may be associated with COPD exacerbations. For example, exhaled condensate levels of leukotriene B4 and 8-isoprostane in COPD patients have been found to be elevated during acute exacerbations. Accordingly, in some variations the measurement device may be configured to measure exhaled condensate levels of these markers of interest. H202 may be another marker of interest for exhaled breath condensate.

In one variation, a patient's exhaled condensate may analyzed on a frequent basis (at least once weekly, if not daily) in order to track changes in a suitable marker of interest, such as leukotriene B4, 8-isoprostane, and/or H2O2.

Exhaled nitric oxide and/or carbon monoxide. Another variation of the system includes a measurement device that can measure the fractional exhaled nitric oxide and/or carbon monoxide of a patient remotely (at home) on a frequent basis (at least once weekly or at least daily) and transmit that data remotely, alone or in combination with other biometrics measured remotely. Fractional exhaled nitric oxide (FENO) may be a pulmonary biomarker in chronic obstructive pulmonary disease (COPD) and/or other respiratory conditions. FENO may be a strong predictor of sputum eosinophilia in patients with COPD exacerbations with sputum eosinophilia.

Volatile organic compounds in breath. In some variations, the patient's exhaled breath may be further analyzed for exhaled biomarkers of interest including volatile organic compounds. Data regarding the changes in exhaled biomarkers may be remotely transmitted and used at least in part to detect and predict a respiratory event (e.g., COPD exacerbation). Exemplary exhaled biomarkers include Cyclohexanone; n-butane; 4-heptanone; 2-pentanone; n-heptane; methylpropyl sulfide; dimethyl di sulfide (DMDS); 6-methyl-5-heptene-2-one; 2,4-dimethylheptane; 2,6-dimethyloctane; cyclohexane; 2-methylhexane, or other suitable biomarkers.

Frequent VOC analysis (such as daily) can be done in the patient home to remotely track changes in VOC composition as a predictor of future acute COPD exacerbation, alone or in conjunction with other biometrics. For example, a patient may collect a daily exhaled sample in a device chamber. The device chamber either has nanosensors embedded, or the device chamber is placed in a different device in the patient home which is an analyzer with sensors. The exhaled breath may be analyzed for presence of cyclohexanone; n-butane; 4-heptanone; 2-pentanone and/or other VOCs. The results of the VOC analysis may be uploaded remotely along with one or more other biometrics of the patient's daily assessment, for example changes in oxygen saturation or breath sounds.

In some variations, analysis of VOC is done via thermal desorption-gas chromatography-time of flight-mass spectrometer, gas chromatography—mass spectrometry, sensor array systems (eNoses), or other VOC analysis methods known in the art.

Respiratory sounds. In some variations, the measurement device may include one or more microphones 220. For example, at least one microphone may be positionable for recording respiratory sounds. For example, at least one microphone may be positioned on the patient's suprasternal notch to record sounds while the patient breathes. Another one or more microphones may be used to simultaneously record ambient noise, and signal processing may cancel out ambient noise from the recorded respiratory sound waveform. In some variations, the recordation of respiratory sounds may be triggered by skin contact. For example, one or more capacitive sensors may allow for detection of when the patient places a microphone portion of the measurement device on skin, which may cause the recording of respiratory sounds to begin. Similarly, the one or more capacitive sensors may allow for detection of when the patient removes the microphone portion of the measurement device from skin, whereupon recording of respiratory sounds may cease.

Recordation of respiratory sounds may be interlocked with the measurement of other bodily metrics, such that obtaining respiratory sounds and obtaining one or more other bodily metrics are mutually dependent on one another. For example, a patient may place his or her finger on a finger-receiving surface of a pulse oximeter portion of a measurement device, which may subsequently cause a microphone to turn on recording. The patient may place a microphone portion of the device on his or her suprasternal notch and breathe. When the patient removes his or her finger from the pulse-oximeter portion of the device, a microphone may cease recording. Such variations may allow for simultaneous capture of oxygen saturation and respiratory sounds.

As described in further detail below, changes in respiratory sounds and/or other bodily metrics can be classified (e.g., using a suitable trained machine learning model), thereby supporting in the prompt detection and treatment of COPD exacerbations and/or other respiratory events.

Respiratory rate. In some variations, the measurement device may be configured to measure respiratory rate. For example, the measurement device may provide for impedance pneumography, capnography, and/or include a pulse oximeter providing a photoplethysmogram (PPG). Respiratory rate may be derived from one or more of these metrics. As another example, the measurement device may include one or more microphones 220 positionable for recording respiratory rate (e.g., as described above with respect to recording respiratory sounds). For example, the patient may recite a known excerpt of speech (e.g., a standard reference sentence, phrase, paragraph, or the like) into the microphone during each assessment or gathering of bodily metrics. From a recording of such recitation (e.g., via application of suitable filters, etc.), respiratory rate may be assessed.

Conventional assessments of how to incorporate respiratory rate to assessing respiratory events have led to mixed results. For example, the works of Yanez et al. (“Monitoring breathing rate at home allowed early identification of COPD exacerbations” Chest. 2012 December; 142(6): 1524-1529) found an increase in the mean respiratory rate one to five days prior to hospitalization due to an acute COPD exacerbation among studied patients. In contrast, Martin-Lesende (“Impact of telemonitoring home care patients with heart failure or chronic lung disease from primary care on healthcare resource use (the TELBIL study randomized controlled trial)” BMC Health Serv. Res. 2013 Mar. 28; 13:118) found no significant change in respiratory rate five days before hospitalization. However, in some variations, respiratory rate may be one of multiple bodily metrics measured and analyzed in the system. By capturing and analyzing respiratory rate along with other bodily metrics, the system may increase overall predictive value.

Cough frequency and/or quality. Chronic cough is a key feature of COPD and variations of cough are known in the art (for example productive vs. non-productive cough). Increase in cough is typically a patient reported symptom as part of a COPD exacerbation. Frequency of daily cough count may improve as a patient recovers from a COPD exacerbation using an ambient sound recorded. In one variation, the system records a patient's hourly or daily cough frequency. Recording may be done via auditory recording (e.g., similar to respiratory sounds as described above), or inferenced based on positional changes, vibrational changes, or other methods known in the art. Furthermore, in some variations, nocturnal cough may be monitored.

Vocal quality. In some variations, deteriorations and/or other changes of vocal quality may be recorded and analyzed for prediction of likelihood of a respiratory event. For example, the measurement device may include one or more microphones 220 as described above with respect to respiratory sound recordation. In some variations, the patient may recite a known excerpt of speech (e.g., a standard reference sentence, phrase, paragraph, or the like) into the microphone during each assessment or gathering of bodily metrics. Accordingly, changes or overall quality of the patient's voice may be analyzed as a factor to predict likelihood of a respiratory event.

Accelerometer with step counting and/or positional data. In some variations, the measurement device may include a sensor, such as an accelerometer, which can track patient physical activity or positional data (e.g., pedometer, distance traveled, etc.). Level of exercise tolerance and daily physical activity may be correlated with risk of hospitalization and mortality in COPD and other respiratory conditions. Furthermore, in some variations the accelerometer may incorporated into a remote pulmonary rehabilitation program that is initiated through the disclosed system. For example, if a patient is inactive, the system may remind the patient, caregiver, or healthcare team that the patient has not had any activity for a certain time period, and in response, the patient is assessed for clinical status or the patient is encouraged to increase physical activity, such as walking around the house or using a stationary bicycle, and the activity can be tracked using sensors in the system, such as with an accelerometer. Physical activity trackers can be incorporated into the system to help patients perform remote pulmonary rehabilitation exercises.

Impedance (resistance). Some variations of the disclosed system provide sensors that can measure thoracic impedance in patients with COPD. Impedance, which is the biological equivalent of resistance, is Ohm's law: R=V/I. Resistance (R) is a function of the relationship between the applied voltage (V) and the current (I) measured across an electric field. Impedance has been used as a measure to predict worsening heart failure episodes. In heart failure, a congested chest has higher fluid content and thus a lower resistance (or impedance), as water is a better electric conductor than air. In COPD, where a patient may have increased air trapping, the opposite may be observed. In addition, a significant proportion of all cause 30-day readmissions for COPD patients is due to episodes of heart failure, as a portion of COPD patients have chronic heart failure (CHF) as a co-morbidity. In addition, thoracic impedance may help distinguish between various causes of changes in patient biometrics. For example, an increase in respiration rate and heart rate with a stable thoracic impedance might warn of a chronic obstructive pulmonary dis- ease exacerbation instead of a heart failure exacerbation.

Central venous pressure estimation. Increases in Central Venous Pressure (CVP), which in many cases is synonymous with Right Atrial Pressure (RAP), occurs in patients with exacerbation of heart failure, along with other causes. Remotely measuring changes in CVP or RAP may help distinguish between an exacerbation for COPD vs. heart failure in patients who are co-morbid with both conditions. For example, one method to measure CVP is measuring the velocity of tricuspid valve using Doppler ultrasound in order to mathematically approximate CVP.

In one variation, a non-invasive measurement device, such as a Doppler ultrasound, may be used to remotely measure velocities of the tricuspid valve to approximate CVP or RAP on a frequent basis (at least once weekly) and that data is transmitted along with other relevant information for COPD patients who may or may not have a co-morbidity of heart failure.

In another variation, a subcutaneously placed device can measure velocities of the tricuspid valve to approximate CVP or RAP on a daily or continuous basis and that subcutaneous device can also measure and transmit one or more biometrics described in this disclosure, for example oxygen saturation, breath sounds, heart rate, respiratory rate, carbon dioxide saturation, and/or other biometrics.

Impulse Oscillometry/Forced Oscillation Technique. Impulse oscillometry (IOS) is a variant of forced oscillation technique (FOT), which permits passive measurement of lung mechanics. In this method, sound waves are superimposed on normal tidal breathing, and the disturbances in flow and pressure caused by the external waves are used to calculate parameters describing the resistance to airflow and reactive parameters that mostly relate to efficient storage and return of energy by the lung. It requires minimal patient cooperation and can be done easily in subjects who are unable to perform spirometry. Importantly, IOS can differentiate small airway obstruction from large airway obstruction and is more sensitive than spirometry for peripheral airway disease IOS parameters seem to be able to pick up early changes in lung function.

In FOT, the sound waves, generated with the help of a loudspeaker are transmitted into the lungs of the subject. These sound waves, which are essentially pressure waves, cause changes in the pressure and this change in pressure drives changes in airflow. By measuring the magnitude of change in the pressure and flow, one can determine the mechanical properties of the lung. Waves of lower frequencies travel deep into lungs till alveoli and are reflected back while those of higher frequencies are reflected from the larger airways. Thus, the parameters calculated at different frequencies can give measures of different regions in the lungs. One main difference is that in FOT, the sound waves of different frequencies were transmitted sequentially, whereas in IOS, an impulse, which can be mathematically decomposed into different frequencies, is transmitted. This helps in reducing the time of test and also provides a high signal to noise resolution.

In some variations, Impulse Oscillometry or Forced Oscillation Technique, is included as part of the system along with measurement of other biometrics included in this disclosure. For example, a device in the disclosed system may have a speaker on a mouthpiece capable of sending an impulse sound wave to collect IOS data. The data collected using impulse oscillometry or forced oscillation technique is measured at least once weekly, and is remotely transmitted alone or in combination with other biometric data. Such data deom FOT/IOS assessment may include but are not limited to the respiratory system impedance (Zrs), ratio of the mouth pressure to airflow according to the impulses, the Rrs, its real component, and the Xrs, its imaginary component, the R5 (Rrs at 5 Hz), R20 (Rrs at 20 Hz), X5 (Xrs at 5 Hz) and Fres (resonant frequency of Xrs).

### Other Features

Additionally or alternatively, in some variations, the measurement device may include an emergency notification feature. For example, in some variations in which the measurement device is a wearable device (e.g., pendant), the measurement device may include a button or other notification feature that, when triggered, can contact a designated care supporter and/or central service provider to hail for assistance in the case of an emergency. For example, if the patient becomes injured (e.g., due to a fall) and is unable to access conventional communication modes, the patient may press the button to notify a designated contact that he or she needs assistance.

### Computing Components

Generally, the measurement device 200 may include a controller including a processor(s) 230 (e.g., CPU) and memory device(s) 240 (which can include one or more computer-readable storage mediums). The processor may incorporate data received from memory and user input. The memory may include store instructions to cause the processor to execute modules, processes, and/or functions associated with the methods described herein. In some variations, the memory and processor may be implemented on a single chip, while in other variations they can be implanted on separate chips.

The processor may be any suitable processing device configured to run and/or execute a set of instructions or code, and may include one or more data processors, image processors, graphics processing units, physics processing units, digital signal processors, and/or central processing units. The processor may be, for example, a general purpose processor, a Field Programmable Gate Array (FPGA), an Application Specific Integrated Circuit (ASIC), and/or the like. The processor may be configured to run and/or execute application processes and/or other modules, processes and/or functions associated with the system and/or a network associated therewith. The underlying device technologies may be provided in a variety of component types (e.g., MOSFET technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and/or the like.

In some variations, the memory may include a database and may be, for example, a random access memory (RAM), a memory buffer, a hard drive, an erasable programmable read-only memory (EPROM), an electrically erasable read-only memory (EEPROM), a read-only memory (ROM), Flash memory, and the like. The memory may store instructions to cause the processor to execute modules, processes, and/or functions such as measurement data processing, measurement device control, communication, and/or device settings. Some variations described herein relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also may be referred to as code or algorithm) may be those designed and constructed for the specific purpose or purposes.

Examples of non-transitory computer-readable media include, but are not limited to, magnetic storage media such as hard disks, floppy disks, and magnetic tape; optical storage media such as Compact Disc/Digital Video Discs (CD/DVDs); Compact Disc-Read Only Memories (CDROMs), and holographic devices; magneto-optical storage media such as optical disks; solid state storage devices such as a solid state drive (SSD) and a solid state hybrid drive (SSHD); carrier wave signal processing modules; and hardware devices that are specially configured to store and execute program code, such as Application-Specific Integrated Circuits (ASICs), Programmable Logic Devices (PLDs), Read-Only Memory (ROM), and Random-Access Memory (RAM) devices. Other variations described herein relate to a computer program product, which may include, for example, the instructions and/or computer code disclosed herein.

The network communication interface 250 may, for example, include a wired connection (e.g., physical connection such as a cable with a suitable connection interface such as USB, mini-USB, etc.) or a wireless network (e.g., through NFC, Bluetooth, WiFi, RFID, or any type of digital network not connected by cables). Some wireless network deployments may combine networks from one or more multiple cellular networks (e.g., 3G, 4G, 5G, etc.) and/or use a mix of cellular, Wi-Fi, and satellite communications.

### User Computing Device

One or more bodily metrics may be user-entered instead of measured by one or more sensors. In some variations, the user-entered bodily metrics may be entered through an interface on the measurement device (e.g., keyboard or display screen interface) and/or a docking station as described below. Additionally or alternatively, such metrics may be entered through a user computing device. Exemplary user computing devices include a mobile phone, tablet, personal computer, or other suitable handheld computing device.

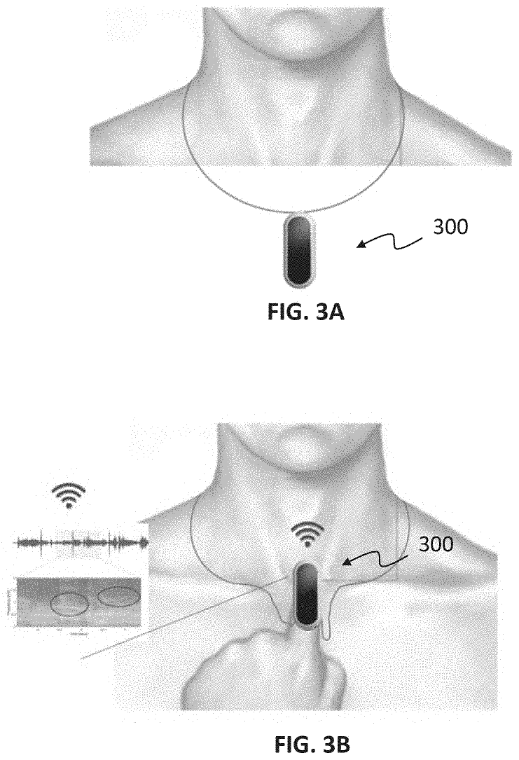

For example, as shown in FIG. 5, an exemplary variation of a user computing device 500 may include at least one network communication interface 510, at least one processor 520, at least one memory device 540, at least one audio device 540, and/or at least one display device 550. The network communication interface 510, processor 520, and memory device 540 may be similar, for example, to those described above with respect to the measurement device. However, in a user computing device 500, the memory device 540 may further be configured to store a mobile application program, or monitoring application 532, which may provide a user interface for a patient to enter bodily metrics such as weight, temperature, illnesses. The mobile application may furthermore be configured to display a history of patient metrics for self-tracking or monitoring.

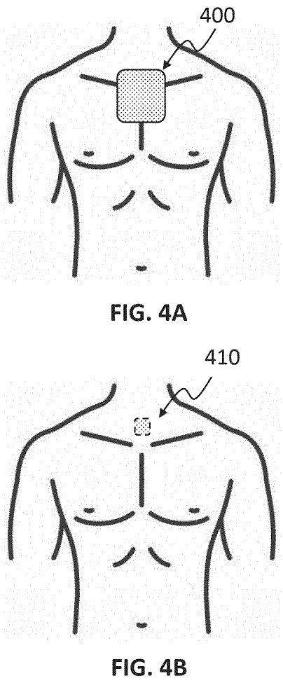

Additionally or alternatively, the mobile application may provide a user interface for a patient to enter patient-reported metrics. For example, as shown in FIG. 6A, an exemplary GUI 600 for receiving patient-reported systems may be displayed on a user computing device having a network communication interface for transmitting the patient-reported metrics. Specifically, the GUI 600 may receive scores characterizing severity in breathlessness, cough, and/or sputum/mucus, such as using the breathless, cough, and sputum scale (BCSS) or suitable variations thereof. The BCSS is a three-item questionnaire rating breathlessness (FIG. 6B), cough (FIG. 6C) and sputum/mucus (FIG. 6D) on a 5-point Likert scale from 0 (no symptoms) to 4 (severe symptoms). However, alternatively, the severity scores may be have any suitable range. Generally, there may be rise in BCSS in days leading up to a COPD exacerbation and/or other respiratory or other clinical events. Thus, use of BCSS may be used to assess likelihood of an upcoming respiratory or other clinical event, as further described below.

The mobile application (or other feature of the user computing device) may be configured to prompt the patient to complete other symptom questionnaires. For example, other COPD symptom questionnaires/assessments include, but are not limited to, St. George's Respiratory Questionnaire, COPD Assessment Test (CAT), scoring of sputum based on volume, frequency of expectoration ≥50%, sputum purulence, or Modified Medical Research Council Dyspnea Scale (mMRC), or EXAcerbation of Chronic Pulmonary Disease Tool (EXACT).

In one variation, the system may collect BCSS or other COPD questionnaires from a patient at least once daily. An alarm may trigger the patient to complete the BCSS or other questionnaire on the patient user computing device or other device capable of remotely transmitting data. Furthermore, a pre-recorded voice or chatbot may be configured to ask the patient questions to conduct a BCSS or other COPD questionnaire. Additionally or alternatively, the patient may be called via phone to answer the BCSS verbally.

### Docking Station

In some variations, the system may include a docking station for receiving the measurement device (and/or user computing device, or other measurement devices). Generally, the docking station may provide a base for downloading patient data from the measurement device (e.g., if the measurement device does not have its own network communication interface for transmitting patient data to a server), recharge a power source of the measurement device, and/or obtain other bodily metrics through one or more sensors on the docking station and/or through a user interface. The docking station may include any of the one or more sensors described above for the measurement device. The docking station may be suited, for example, for bedside placement or similar location, where the docking station may be configured to record nocturnal data (e.g., nocturnal cough) while the patient is sleeping, recharge the measurement device while not being worn by the sleeping patient, be in a convenient location for providing an audible, visual, or tactile alarm notification as a reminder for the patient to perform an assessment (e.g., upon waking up), etc.

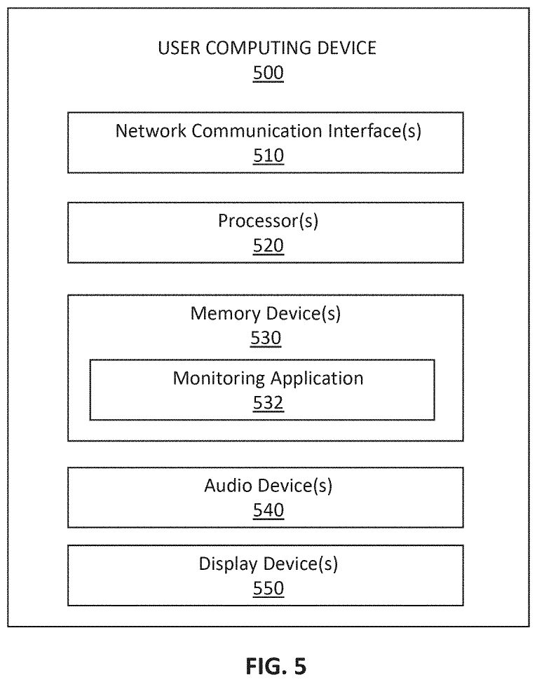

In some variations, as shown in the illustrative schematic depicted in FIG. 7, a docking station 700 may include a housing including a receptacle 702 that is configured to removably receive a measurement device having one or more sensors for measuring one or more bodily metrics of the patient (e.g., measurement device as described above). A power supply 760 in the docking station may be configured to recharge a power source of the measurement device when the measurement device is docked in the docking station.

Furthermore, the docking station 700 may include other computing devices, such as at least one network communication interface 710, at least one processor 720, at least one memory device 730, and/or at least one audio device 740 (e.g., microphone and/or speaker device). These devices may be similar to those described above with respect to the measurement device. However, in the docking station 700, the one or more memory devices 730 may further be configured to store a monitoring application 732 providing for operation of the docking station as described herein. Furthermore, the one or more memory devices 730 may be configured to receive and store bodily metric data from the one or more sensors of the measurement device. The docking station 700 may further include at least one network communication interface 710 (e.g., wireless communication module) configured to transmit the stored bodily metric data. A user interface 750 may allow a user to provide input to the docking station 700, such as for changing settings of the docking station and/or measurement device (e.g., time settings for the alarm notification), entering patient-reported bodily metrics (e.g., BCSS scores), changing a displayed clock time on a display, etc.

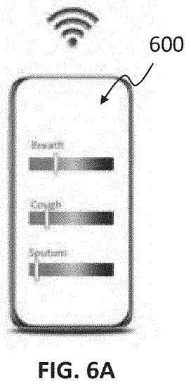

An exemplary illustrative variation of a remote monitoring system 800 including a docking station 820 is shown in FIGS. 8A and 8B. As shown in FIG. 8A, a measurement device 810 may be removably received in the docking station 820 (FIG. 8B). For example, the docking station 820 may include a cavity or recess that is shaped in a complementary manner to the measuring device 810. The docking station may include a locking element (e.g., snap fit, latch, etc.) to secure the measurement device in the cavity or recess. Upon the measurement device being received in the docking station 820, a power supply in the docking station 820 may be configured to begin recharging a power source of the measurement device. Furthermore, patient data from the measurement device may be downloaded to one or more memory devices of the docking station 820, and transmitted through a network communication interface. The charging and/or patient data transmission may be automatically performed upon detection of the docking of the measurement device in the docking station, and/or initiated with an affirmative action by the user (e.g., pressing a button, selecting a suitable icon on the display 840, etc.

Although the housing 820 shown in FIGS. 8A and 8B is generally semi-circular (or hemispherical), it should be understood that the housing may have any suitable shape, such as prismatic (e.g., rectangular prismatic). The housing may have a flat bottom surface to facilitate placement on a bedside surface such as a nightstand, and/or other suitable attachment features such as a hanging strap, suction cup, fastener interface, etc. for hanging on a bedpost and/or attaching to a wall surface.

The docking station 820 further includes various user interface elements including a symptom entry interface 830, a display 840, and a settings control element 850 (e.g., knob, button, switch, etc.). The symptom entry interface 830 may include an array of keys, buttons, selectable displayed icons, or the like that correspond to options for entry of patient-reported bodily metrics. For example, as shown in FIGS. 8A and 8B, the symptom entry interface 830 may include selectable numerical elements corresponding to BCSS scores (i.e., 0, 1, 2, 3, 4).

The settings control element 850 may be a hardware component suitable for adjusting one or more settings of the docking station and/or measurement device. For example, the settings control element 850 shown in FIGS. 8A and 8B may be configured to adjust the time for an alarm notification to the patient for performing an assessment. However, the settings control element 850 may be configured to control any suitable feature.

The display 840 may be configured to display any suitable digital information, such as clock. In some variations, the display 840 may be further configured to display a suitable graphical user interface (GUI), such as patient data or other information, operational status of the docking station and/or measurement device (e.g., charging status), other settings (e.g., alarm volume), status of portions of a bodily metrics assessment completed. Furthermore, although the symptom entry interface 830 and settings control element 850 are depicted in FIGS. 8A and 8B as separate elements from the display 840, it should be understood that in some variations, the functionalities of these elements may be integrated with the display 840. For example, the display 840 may be configured to display a GUI that includes a navigable menu for a patient to enter symptoms or other patient-entered bodily metrics, and/or for a patient to adjust the time for an alarm notification, etc.

In some variations, the docking station may further be configured to provide other useful features such as a clock face or time display, an alarm independent of the above-described alarm notification for a health assessment, connectivity to a telephone and/or video chat feature, connectivity to social media applications and/or third-party smart assistants, charging ports for a user computing device and/or other personal electronic devices, etc. Furthermore, the docking station may include readily accessible storage for medication (e.g., inhaler device), such as a drawer or other suitable compartment.

### Other Devices

Air quality monitor. In some variations, the system may include an air quality monitor. The air quality monitor may be standalone, or may be incorporated in another device described herein (e.g., docking station, measurement device, etc.). Air quality, temperature, and humidity, may be potential triggers to COPD exacerbations. Air pollutants include but are not limited to sulfur dioxide (SO₂), nitrogen dioxide (NO₂), and particulate matter <2.5 microns (PM_{2.5}). In some variations, the system includes data collection of air quality, air temperature, and air humidity in the patient's surroundings (such as in the home). The sensor may also detect smoke associated with cigarettes to track whether a patient is continuing to smoke or is exposed to second hand smoke. In other variations, air quality, temperature, and humidity data of known locations, such as patient zip code, may be used to collect and analyze data in conjunction with biometric data to provide COPD exacerbation risk assessment. For example, real-time continuous outdoor air measurements, reported as hourly averages may be pulled from USEPA's Air Quality System (AQS) for SO₂, NO₂ and PM_{2.5}. Data for important meteorological variables (e.g., temperature and humidity) can be exported as 24-h daily averages from the National Oceanic and Atmospheric Administration's (NOAA) National Climatic Data Center (NCDC). Data on regional trends of influenza can be obtained from the US Outpatient Influenza-like Illness Surveillance Network (IliNet), available from the Center for Disease Control's (CDC) FluView application.

Supplemental oxygen monitor. Additionally or alternatively, in some variations, the system may include a sensor that records the level of supplemental oxygen used by a COPD patient and remotely transmits this data, alone or along with the patient's oxygen saturation level and/or carbon dioxide saturation level. Such data may be transmitted regularly or on a frequent basis (at least weekly or at least daily). Additionally or alternatively, amount and/or rate (e.g., L/min) of supplemental oxygen consumed by the patient may be reported (e.g., over an automated phone system such as that described below).

Current clinical guidance recommends that all patients with respiratory failure in the context of a diagnosis or history suggestive of COPD receive oxygen therapy targeted to 88%-92% until hypercapnia has been excluded by arterial blood gas analysis. Frequently, COPD patients arrive to the hospital with supplemental oxygen levels too high, which can cause increased CO2 (hypercapnia) due to complex physiological reasons. Patients may fall short of breath and increase the oxygen level beyond the target of 88%-92%, or this may be done by paramedics prior to patient arriving at hospital. Thus, for patients with COPD, it is important to titrate the level of home oxygen between 88%-92%. An increase use of supplemental oxygen to maintain the 88%-92% may be a signal of an impending COPD exacerbation.

Peak expiratory flow. The peak expiratory flow, also called peak expiratory flow rate (PEFR) is a person's maximum speed of expiration, as measured with a peak flow meter, a small, hand-held device used to monitor a person's ability to breathe out air. In asthma patients, peak expiratory flow rate (PEFR) measurement has been promoted as a useful tool for assessing airway obstruction and titrating therapy. It is not only cheap, but has high reproducibility and user compliance rates, making it a useful tool for ambulatory monitoring of asthma. In COPD, however, the utility of PEFR monitoring remains unclear. COPD patients may have greater variability in daily peak expiratory flow reduced time to first hospitalization, more frequent hospitalizations, and higher all-cause mortality despite similar demographic, spirometric and comorbid parameters at baseline. For example, Seemungal et al. (“Time course and recovery of exacerbations in patients with chronic obstructive pulmonary disease” Am J Respir Crit Care Med. 2000 May; 161(5):160-13) describe that in a study of asthma exacerbations, peak flow readings fell and symptom scores associated with exacerbation increased for approximately 7 days before exacerbation, in contrast to COPD exacerbations where there were no peak flow or FEV, changes before onset of exacerbation, but patient symptoms increased before exacerbation. Seemungal et al. describe that peak expiratory flow or FEV, monitoring in patients with COPD to detect the early development of an exacerbation may not be useful in clinical practice.

In some variations, the system includes a device to monitor peak expiratory flow in COPD patients. Patients who show variability in PEFR may be at higher risk for exacerbation. Tracking PEFR in patients who are discharged from hospital or recovering from exacerbation may provide utility as patients whose PEFR does not recover as predicted are higher risk for re-admission to the hospital. In some variations, the system includes a device to remotely monitor peak expiratory flow in COPD patients on a frequent basis (at least weekly or daily) and data of PEF is transmitted remotely alone or in combination with other biometrics in this disclosure. In some variations the PEFR sensor is part of device that has pulse oximetry and a digital stethoscope.

Spirometry. In some variations, a spirometer with ability to remotely transmit data is used by a patient frequently (at least weekly if not at least once daily) to capture spirometry patterns and transmit data remotely. Measurements may include but are not limited to vital capacity (VC), Forced vital capacity (FVC), Forced expiratory volume (FEV) at timed intervals of 0.5, 1.0 (FEV1), 2.0, and 3.0 seconds, forced expiratory flow 25-75% (FEF 25-75) and maximal voluntary ventilation (MVV) or other parameters of spirometry.

Photo/audio/video of patient (or sputum/mucus or other patient features). In some variations, the system may allow the patient to record and remotely transmit data of the patient or patient features. For example, a patient may use the measurement device (or user computing device, described below) to take a photo/video of symptoms (such as edema), or an audio/visual recording, or take a photo/video of sputum/mucus. Machine learning and/or other process may be used to analyze such data, for example, using the image to predict whether sputum is purulent, in order to guide potential therapy decisions. The patient may also request a virtual check-in with a health care provider at any time and use the system to send a message for such a request. Video of the patient could be used to determine patient risk for needing to seek immediate medical attention in-person vs. ability to be treated remotely at home.

Weight. Part of the system may include a weight scale that captures the patient's weight and transmits that data to be analyzed. For example, in one variation the weight scale may have cellular connectivity (3G, LTE, etc.) or be connected via Wi-Fi or Bluetooth to transmit weight.

Blood pressure. In some variations, part of the system may include a digital blood pressure assessment where blood pressure is collected and transmitted as part of the system. Alternatively blood pressure may be manually measured and entered by the patient or a medical care provider (e.g., through a user computing device, over the phone as described below, etc.).

Temperature. Additionally or alternatively, COPD patients with a fever may be at higher risk of a COPD flare. Some variations include a sensor that can capture and transmit data related to a patient's body temperature. In some variations, temperature is part of the daily assessment of the system. Additionally or alternatively, a healthcare team member may utilize the system to request a patient capture their temperature data on request.

Compliance. In some variations, the system may include one or more sensors on a patient's inhaler or pill bottles in order to record/track when a patient uses an inhaler or may take a pill/tablet and remotely transmit that data as part of the disclosed system. For example, data in the system that has missing compliance data and change in certain biometrics (for example respiration rate or O₂ saturations) may suggest a patient at higher risk for medical follow-up.

### Telephone Assessment

Additionally or alternatively, at least some of the patient data (e.g., any of the bodily metrics described above) may be provided by a patient through another suitable user interface, such as dictated over the phone during a phone call with an automated phone system, operator, etc. or through a mobile application executed on a user computer device. For example, patient data may be communicated at least in part over a remote condition management platform as described above with respect to FIGS. 1A-1D. For example, an automated phone system (or operator) may ask the patient questions or otherwise prompt the dictation of patient data and/or a reference excerpt of speech (as described above). The voice may be transcribed (e.g., automatically by the automated voice system or manually by the operator) into text and parsed into relevant patient data (e.g., specific bodily metrics). In some variations, a patient may additionally or alternatively enter at least some patient data via a touchpad (e.g., keypad on a telephone). In some variations, the patient may be instructed to call the automated phone system and/or operator by a certain time every day (e.g., 10 AM), and failure to do so may prompt the automated phone system or other operator to separately contact the patient and/or medical care provider 140 for follow-up. Accordingly, the automated phone system may function to gather patient data (e.g., bodily metrics) that are recited or dictated over the phone. For example, in an illustrative implementation, the patient may call into an automated phone system and report bodily metrics including heart rate, SpO₂, and BCSS score (and/or individual scores of breathlessness severity, cough severity, and sputum severity). However, in other examples, any of the above-described bodily metrics (e.g., blood pressure) may be measured with a suitable separate device (e.g., pulse oximeter) and reported over the telephone during an assessment call to the automated phone system.

Additionally or alternatively, the patient may recite a speech excerpt (e.g., sentence, paragraph, etc.), as described above, for recordation. The recordation may be processed, for example, to assess respiratory sounds, respiratory rate, vocal quality, and the like, as described above. For example, a respiratory rate and/or respiratory sounds may be derived from breath sounds and/or duration of a phone call while the patient is on the phone after calling into an automated phone system as described above, and/or by analyzing a speech excerpt. Furthermore, the patient may be asked to provide other suitable information (e.g., in response to prompting questions), such as current level of supplemental oxygen being used (e.g., in liters/min).

Furthermore, the selection of bodily metrics for reporting may differ depending on stage of patient condition. For example, in some variations, the system may specifically configured for some patients who are discharged from the hospital after a COPD exacerbation and may benefit from remote monitoring of a wider suite of bodily metrics. For example, a patient discharged from the hospital after a COPD exacerbation may additionally have his or her blood pressure remotely monitored (e.g., an automated phone system may prompt the patient to report his or her blood pressure during an assessment).

Even further, the selection of bodily metrics for reporting may differ depending on the respiratory condition being monitored. As an illustrative example, in some variations, the system may be configured to monitor asthma patients. In these variations, the patient may, for example, be provided with a peak expiratory flow rate (PEFR) meter, and during the assessment (e.g., daily assessment) the patient may report PEFR value as measured by the PEFR meter, along with asthma-related symptoms. An example of asthma assessment is described in further detail below.

### Predictive Analysis System

Generally, a predictive analysis system may receive and analyze the patient data (e.g., bodily metrics) from the measurement device, user computing device, docking station, and/or any other device. Exemplary details of such analysis are described in further detail below. As shown in

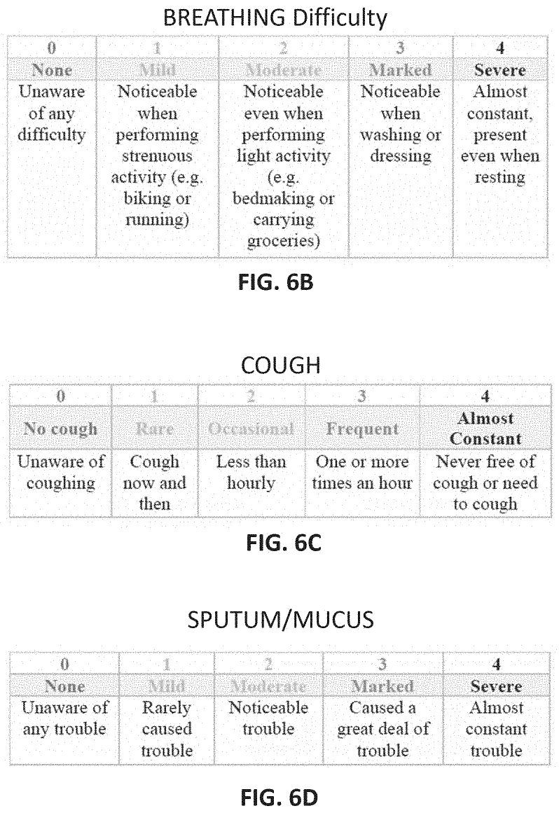

FIG. 9, a predictive analysis system 900 may include at least one network communication interface 910 for receiving bodily metrics, at least one processor 920, and at least one memory device 930 for storing received bodily metrics. These components may, for example, be similar to those described above with respect to the measurement device. Furthermore, the one or more memory devices may be configured to store one or more software modules for analyzing the received and stored bodily metrics. For example, the memory device(s) 930 may include a predictor module 932 configured to assess likelihood of an upcoming respiratory event, such as with a predictive algorithm. Additionally or alternatively, the memory device(s) 930 may include a learning module 934 configured to train and/or apply a machine learning model for assessing likelihood of an upcoming respiratory event, as further described below.

### Methods for Remotely Managing a Chronic Respiratory Condition

Generally, in some variations, a method for managing a chronic respiratory condition of a patient includes receiving a plurality of bodily metrics for a patient over a remote communication link, predicting the likelihood of an upcoming respiratory event for the patient based on the plurality of bodily metrics, and communicating an alert to a medical care provider (e.g., pulmonologist, other physician or nurse practitioner, other medical staff, etc.) based at least in part on the predicted likelihood of an upcoming respiratory event.

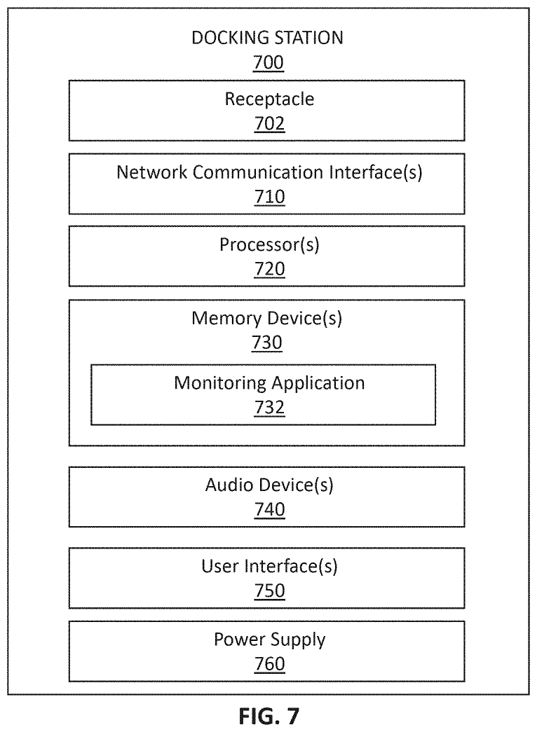

FIG. 10 illustrates a general workflow of processes involved in a method for remotely managing a chronic respiratory condition, performed among a patient 110, a predictive analysis system 130, and one or more medical care providers 140. The workflow may be similar to processes for the remote condition management platform described above with respect to FIGS. 1A-1C, and further described below with respect to management of a respiratory condition.

### Generating Patient Data

Patient data including bodily metrics may be obtained in any of various manners. In some variations, as shown in FIG. 10, an alarm notification (1010) may be provided to the patient as a reminder to generate patient data in an assessment. Alternatively, in some variations, the alarm notification may be omitted. The alarm notification may be provided repeatedly over a monitoring period, such as once daily, twice daily (e.g., in the morning and at night), etc. The alarm notification may be provided at a consistent and/or user-selected time of day, or any convenient time as described in further detail above.

Upon receiving the alarm notification as a prompt to perform an assessment, the patient may generate patient data (1020) by facilitating measurement of metrics by one or more sensors (e.g., oxygen saturation, heart rate, etc. as described above) and/or entering patient-reported symptoms or other metrics (e.g., BCSS scores) through the system. Suitable exemplary patient data (e.g., bodily metrics) is explained in further detail above. If the patient fails to perform the assessment within a predetermined period of time after receiving the alarm notification (e.g., 1 minute, 2 minutes, 5 minutes, 10 minutes, etc.), then the alarm notification may be provided again and/or a designated care supporter or other contact may be notified to follow-up with the patient. The patient data may be provided (1022) to the predictive analysis system, such as through a suitable wired or wireless network communication interface.

In another variations, a patient may call into a phone system (e.g., automated phone system, operator-manned phone system, etc.) to report assessments on a periodic (e.g., daily) basis. For example, data transmission of heart rate, SpO₂, BCSS score, and voice data (e.g., recitation of a reference speech excerpt) may be collected via an automated phone system, and subsequently transmitted, recorded, and stored on a server (e.g., HIPAA-compliant server or other HIPAA-compliant method). In some variations, when the patient calls into a phone system from a particular phone number, the identity of the patient may be automatically confirmed by confirming that the call was made from a predetermined contact phone number (e.g. among the contact phone number provided by the patient during patient setup and onboarding), thereby preventing third parties from reporting fraudulent data. Additionally or alternatively, a patient may be associated with a unique code (e.g., patient serial ID code) that the patient can recite or type (e.g., on phone touchpad) to confirm his or her identity during assessment.

In some variations, the phone system may prompt the patient to answer (through recitation, touchpad entry, etc.) one or more adaptive questions in response entry of one or more bodily metrics. For example, if a patient's trending index score (discussed in further detail below), satisfy a predetermined threshold, additional questions may be asked to better rank the patients, and/or obtain additional clinical context for the medical care provider. For example, adaptive follow-up questions could prompt the patient to enter symptoms such as changes in ambulatory distance, chest pain, leg swelling, fever or chills, changes in medication, or the like. Accordingly, adaptive questioning may elicit additional patient data for use in predictive analysis.

### Analyzing Patient Data

As shown in FIG. 10, the predictive analysis system may receive the patient data (1030), store the data, and proceed to perform analysis on the patient data in order to predict likelihood of an upcoming respiratory or other clinical event (1040), and/or to otherwise characterize severity of medical condition or patient risk, and flag patients suitable for further medical attention (e.g., high-risk patients). Various exemplary methods of performing this analysis are described below.

### Patient Tiering

In some variations, monitored patients may be triaged or sorted into tiered groups (e.g., high-risk, moderate-risk, low-risk, etc.). For example, patients may be sorted into an “alerts” list of patients, or into a “watchlist” of patients corresponding to their likelihood of requiring medical attention. Patients on the alerts list may be considered more likely to require medical attention, compared to patients on the watchlist, and patients on the watchlist may be considered more likely to require medical attention than patients on neither the alerts nor watchlist. Such tiered patient grouping may help reduce alert fatigue by preventing excessive numbers of alerts communicated to a medical care team. Furthermore, such tiered patient groups may help medical care teams focus their attention more effectively to those patients requiring more urgent medical attention.

For example, in some variations, one or more bodily metrics and/or other patient data may be compared to threshold values to help sort or triage patients into patient tiers. The threshold values may include default (e.g., recommended) values, and/or one or more threshold values may be adjusted by a medical care practitioner or other administrator, as described elsewhere herein. Furthermore, one or multiple “rules” may be defined to sort a patient into a particular tier, where each “rule” includes a respective set of threshold values. In other words, a first rule may be based on a first set of threshold values, a second rule may be based on a second set of threshold values, etc., and a patient whose patient data satisfies any one or more of these rules for a particular tier may be placed into that tier. As further described below, placement of a patient into a particular tier may trigger one or more action items or events assisting in patient care.

For example, tiering may be based on one or more rules incorporating heart rate (HR), oxygen saturation (SpO₂), and BCSS scores. As an illustrative example, a patient may trigger an alert (e.g., placed on an “alerts” list) if he or she satisfies any one or more of four alert rules. According to a first alert rule (i), an alert may be triggered in response to absolute BCSS score ≥5 and ≥2-point relative BCSS increase over previous 1 or 2 assessments (e.g., “BCSS rel points” described below). According to a second alert rule (ii), an alert may be triggered in response to absolute BCSS score ≥8. According to a third alert rule (iii), an alert may be triggered in response to SpO₂<88% and absolute BCSS score ≥4. According to a fourth alert rule (iv), an alert may be triggered in response to HR>120 bpm and absolute BCSS score ≥4. In some variations, the method may include triggering an alert if the patient data satisfies any one or more of the alert rules (i)-(iv). Furthermore, it should be understood that these threshold values are exemplary only, and may be adjusted (e.g., based on practitioner or clinic preference).

As another illustrative example, a patient may trigger placement on a watchlist if he or she satisfies any one or more several watchlist rules. According to a first watchlist rule (i), a patient may be placed on a watchlist if he or she satisfied one or more of the alert rules (e.g., as described above) within previous recent assessments (e.g., preceding 1-2 assessments) but whose current patient data from a current patient assessment do not meet any of the alert rules to warrant being placed on the alerts list. According to a second watchlist rule (ii), a patient may be placed on the watchlist in response to absolute BCSS score ≥5 and ≥1-point relative BCSS increase over previous 1 or 2 assessments (e.g., “BCSS rel points” described below). According to a second alert rule (ii), a patient may be placed on the watchlist in response to absolute BCSS score ≥6. According to a third watchlist rule (iii), a patient may be placed on the watchlist in response to SpO₂<88% and absolute BCSS score ≥3. According to a fourth watchlist rule (iv), a patient may be placed on the watchlist in response to SpO₂<86%. According to a fifth watchlist rule (v), a patient may be placed on the watchlist in response to HR>120 bpm and absolute BCSS score ≥3. In some variations, the method may be include placing a patient on the watchlist if the patient data satisfies any one or more of the watchlist rules (i)-(v). Furthermore, it should be understood that these threshold values are exemplary only, and may be adjusted (e.g., based on practitioner or clinic preference).

For example, as shown in FIG. 10, a medical care provider may select suitable alert conditions (1052), such as thresholds and/or other conditions for triggering an alert notification associated with a high-risk patient with a relatively high likelihood of experiencing an upcoming respiratory or clinical event. The alert conditions may be patient-specific (e.g., custom or dependent upon patient characteristics as a whole), demographic-specific (e.g., dependent upon GOLD classification), or default settings. Similarly, a medical care provider may additionally or alternatively select preferred factors for a trending index (as further described below), such as indicating whether to use absolute changes or relative changes in bodily metrics or other settings. For example, a medical care provider may select settings to enable monitoring of a patient based on the patient's changes relative to other patients (e.g., through ranking), and/or monitoring of a patient based on the patient's historical trends (e.g., relative to past bodily metrics).

### Trending Index Score

In some variations, at least some of the received bodily metrics may be aggregated into a trending index score in accordance with a suitable scoring algorithm. The trending index score may, for example, help characterize relative risk (e.g., condition severity) of multiple patients. As described in further detail below, the trending index score may be used to rank patients according to level of risk and/or identify patients for more focused attention in patient tiering (e.g., on an alerts list or watchlist).

Index score based on absolute values. In some variations, the method may include deriving a first, absolute points value for each bodily metric based on the value of the bodily metric, and generating a trending index score based at least in part on these absolute points values. For example, in a first variation, the absolute points values for each bodily metric may be determined based on comparing the value of the bodily metric to one or more predetermined thresholds. The trending index score may be based on the sum of the absolute points values for the plurality of bodily metrics.

In an exemplary illustration of this first variation, the trending index score may incorporate heart rate (HR), oxygen saturation (SpO₂), and BCSS scores. Absolute values and/or relative changes of values of these bodily metrics may be incorporated into determination of the trending index score.

An absolute points value for heart rate may be compared to one or more tiered thresholds, where each tiered threshold corresponds to a respective number of points (“HR abs points”). Higher tiered thresholds may correspond to a greater number of points indicating greater severity of the bodily metric (more likely to indicate a high likelihood of a respiratory event). Exemplary thresholds and corresponding points values are shown below in Table 1.

TABLE 1
Heart rate thresholds for trending index score generation

| | | HR abs |
|---|---|---|
| Tier | Absolute Threshold | points |
| 2 | HR > 100 BPM | 1 |
| 1 | HR > 120 BPM | 2 |

Similarly, oxygen saturation may be compared to one or more tiered thresholds, where each tiered threshold corresponds to a respective number of points (“SpO₂ abs points”). Exemplary thresholds and corresponding points values are shown below in Table 2. Furthermore, in some variations, SpO₂ abs points may be scaled relative to (or otherwise take into account) a baseline SpO₂ for the patient. For example, for a patient whose baseline SpO₂ is around 88%, a SpO₂ measurement of <88% may result in a SpO₂ abs points of 0 (instead of 3 as shown in Table 2), a SpO₂ measurement of <86% may result in a SpO₂ abs points of 1 (instead of 4 as shown in Table 2), etc. In other words, a low measured SpO₂ for a patient having a normally-low baseline SpO₂ may not necessarily result in a low SpO₂ abs points value for that patient.

TABLE 2
SpO₂ thresholds for trending index score generation

| | | SpO₂ abs |
|---|---|---|
| Tier | Threshold | points |
| 3 | SpO₂ ≥ 92% | 0 |
| 2 | SpO₂ < 92% | 1 |
| 1 | SpO₂ < 90% | 2 |
| 1 | SpO₂ < 88% | 3 |
| 1 | SpO₂ < 86% | 4 |
| 1 | SpO₂ < 84% | 5 |

BCSS scores may similarly be compared to one or more tiered thresholds. Specifically, the sum (“BCSS”) of the scores for breathlessness severity (“B abs points”), cough severity (“C abs points”), and sputum/mucus severity (“S abs points”) may be compared to one or more tiered thresholds. Each tiered threshold may correspond to a respective number of points (“BCSS abs points”). Exemplary thresholds and corresponding points are shown below in Table 3. Furthermore, in some variations, a “functional BCSS abs points” value may incorporate an additional increase in the raw BCSS scores (e.g., BCSS abs points +1) to reflect one or more particularly severe component points values (“B abs points”, “C abs points”, “S abs points”), such as if any component points value is at the maximum level of 4 (out of 4). The “functional BCSS abs points” may, for example, be used in place of “BCSS abs points” in the equations below.

TABLE 3
BCSS thresholds for trending index score generation

| | | BCSS abs |
|---|---|---|
| Tier | Absolute Threshold | points |
| 3 | BCSS < 4 | 0 |
| 3 | BCSS = 4 | 1 |
| 2 | BCSS = 5 | 2 |
| 2 | BCSS = 6 | 3 |
| 1 | BCSS = 7 | 4 |
| 1 | BCSS = 8 | 5 |
| 1 | BCSS = 9 | 6 |
| 1 | BCSS = 10 | 7 |
| 1 | BCSS = 11 | 8 |
| 1 | BCSS = 12 | 9 |

In a first variation of generating a trending index score, the trending index score (“TI”) may be the sum of the absolute points values for HR, SpO₂, and BCSS, as shown in Equation 1 below:

TI=(HR abs points)+(SpO₂ abs points)+(BCSS abs points) (1)

Index score based on absolute values and one or more weighting factors. In a second variation of generating a trending index score, the trending index score may be calculated in a manner similar to Equation 1, except with one or more of the absolute points values weighted by an appropriate weighting factor. In other words, the absolute points value associated with at least one bodily metric may be weighted more heavily (e.g., multiplied by a weighting factor) relative to another bodily metric. An exemplary illustration of the second variation of generating a trending index score is shown in Equation 2 below:

TI=(HR abs points)+(SpO₂ abs points)+(weighting factor)*(BCSS abs points) (2)

where the weighting factor associated with the BCSS absolute points value increases the effect that BCSS may have on the final value of the predictive index score. The weighting factor may, for example, be 3, such that the BCSS absolute points value is weighted three times more heavily than the HR absolute points value and the SpO₂ absolute points value.

Index score based at least in part on relative changes. In some variations, the method may include generating a trending index score by deriving a second, relative points value of at least one bodily metric based on the relative change of at least one bodily metric during a monitoring period, and generating a trending index score at least in based on these relative points values (e.g., sum of these relative points values). For example, the relative points value for each bodily metric may be determined based on comparing the relative change of the bodily metric to one or more predetermined thresholds. The trending index score may be based at least in part on the sum of the relative points values for the plurality of bodily metrics. Relative change of a bodily metric may be measured against the immediately prior value of the bodily metric, or across any suitable time interval, such as across the preceding 6 hours, preceding 12 hours, preceding 24 hours, and the like. Alternatively, relative change of a bodily metric may be measured against a running average (mean) for a preceding time interval, such as average across the preceding 12 hours, preceding 24 hours, preceding 2 days, preceding 3 days, etc.

In a third variation of generating a trending index score, the trending index score may be based on the sum of absolute points values and relative points values for each bodily metric. In an exemplary illustration of this variation, the trending index score may incorporate heart rate (HR), oxygen saturation (SpO₂), and BCSS scores (or individual components thereof). Absolute points values (HR abs points, SpO₂ abs points, and BCSS abs points) may be determined as described above. In other variations, solely relative changes in bodily metrics may be used to generate an index score.

The relative change in HR may be compared to one or more tiered thresholds. For example, a greater increase in heart rate since the last measured heart rate value may generally correspond to a higher number of points (“HR rel points”). Exemplary thresholds and corresponding points are shown in Table 4 below.

TABLE 4:
Heart rate thresholds for predictive index score generation

| | | HR rel |
|---|---|---|
| Tier | Relative Threshold | points |
| 2 | HR increase > 10 BPM | 1 |
| 1 | HR increase > 15 BPM | 2 |

Similarly, the relative change in oxygen saturation may be compared to one or more tiered thresholds. For example, a greater decrease in SpO₂ since the last measured SpO₂ value may generally correspond to a higher number of points (“SpO₂ rel points”). Exemplary thresholds and corresponding points are shown in Table 5 below.

TABLE 5
SpO₂ thresholds for trending index score generation

| | | SpO₂ rel |
|---|---|---|
| Tier | Relative Threshold | points |
| 3 | SpO₂ decrease < 2 | 0 |
| 2 | SpO₂ decrease = 2 | 1 |
| 1 | SpO₂ decrease > 2 | 2 |

Relative change in BCSS score (and/or any individual component of BCSS score) may similarly be compared to one or more tiered thresholds. For example, a greater increase in BCSS score (or individual component thereof) may generally correspond to a higher number of points (“Breathing rel score”, “Cough rel score”, “Sputum rel score”). Exemplary thresholds and corresponding points are shown in Tables 6A-6D below.

TABLE 6A
BCSS score thresholds for trending index score generation

| | | BCSS rel |
|---|---|---|
| Tier | Relative Threshold | points |
| 3 | Δ BCSS ≤ 0 | 0 |
| 2 | Δ BCSS = (+1) | 1 |
| 1 | Δ BCSS > (+1) | 2 |

TABLE 6B
Breathlessness severity point thresholds
for trending index score generation

| | | Breathing rel |
|---|---|---|
| Tier | Relative Threshold | points |
| 3 | B abs points change over last 2 day average ≤ 0 | 0 |
| 2 | B abs points change over last 2 day average = (+1) | 1 |
| 1 | B abs points change over last 2 day average > (+1) | 2 |

TABLE 6C:
Cough severity point thresholds for trending index score generation

| | | Cough rel |
|---|---|---|
| Tier | Relative Threshold | points |
| 3 | C abs points change over last 2 day average ≤ 0 | 0 |
| 2 | C abs points change over last 2 day average = (+1) | 1 |
| 1 | C abs points change over last 2 day average > (+1) | 2 |

TABLE 6D
Sputum severity point thresholds for
trending index score generation

| | | Sputum rel |
|---|---|---|
| Tier | Relative Threshold | points |
| 3 | S abs points change over last 2 day average ≤ 0 | 0 |
| 2 | S abs points change over last 2 day average = (+1) | 1 |
| 1 | S abs points change over last 2 day average > (+1) | 2 |

Any of the above variations (e.g., Equations 1 or 2) may be modified with the inclusion of a relative metric score. In one example of the third variation of generating a trending index score, the trending index score may be the sum of various absolute and relative points for HR, SpO2, and/or BCSS score, as shown in Equation 3a below:

TI=(HR abs points)+(HR rel points)

+(SpO₂ abs points)+(SpO₂ rel points)

+(BCSS abs points)

+(BCSS rel points) (3a)

In another example of the third variation of generating a trending index score, the trending index score may be the sum of various absolute and relative points for HR, SpO₂, and/or BCSS score components, as shown in Equation 3b below:

TI=(HR abs points)+(HR rel points)

+(SpO₂ abs points)+(SpO₂ rel points)

+(BCSS abs points)

+(Breathing rel points)

+(Cough rel points)

+(Sputum rel points) (3b)

Index score based at least in part on occurrence of recent alerts. In a fourth variation of generating a trending index score, the trending index score may be based on the sum of absolute points values and/or relative points values for each bodily metric (e.g., similar to the third variation described above), as well as a points value indicating recent alerts. In an exemplary illustration of this fourth variation, the trending index score may incorporate heart rate (HR), oxygen saturation (SpO₂), and BCSS scores (or individual components thereof). Absolute points values and relative points values may be determined as described above. Furthermore, a points value indicating recent alerts may be generated as shown in Table 7 below:

TABLE 7
Recent alert thresholds for trending index score generation

| | | Recent alert |
|---|---|---|
| Tier | #days since meeting alert threshold | points |
| 3 | >2 days | 0 |
| 2 | =2 days | 1 |
| 1 | <2 days | 2 |

Any of the above variations (e.g., Equations 1-4) may be modified with the inclusion of the recent alert points value. In one example of the fourth variation of generating a trending index score, the trending index score may be the sum of various absolute and relative points values for HR, SpO₂, BCSS score, and/or recent alert points value, as shown in Equation 4 below (which is Equation 3a with the addition of the recent alert score).

TI=(HR abs points)+(HR rel points)

+(SpO₂ abs points)+(SpO₂ rel points)

+(BCSS abs points)

+(BCSS rel points)

+(Recent alert points) (4)

In some variations, the trending index score may further be inflated or deflated on other corrective variables. For example, in one variation, if BCSS abs points is greater than some threshold (e.g., 5 or greater) and has increased at a threshold rate (e.g., is at least a 2-point increase over the last 24 or 48 hours), then the trending index score may be incremented (e.g., by 2). As another example, in one variation, if BCSS abs points is lower than the preceding day, then the trending index score may be decremented (e.g., by 1).

Furthermore, the trending index score may additionally or alternatively incorporate other reporting data such as time stamp of an assessment. For example, patients who are not feeling well may tend to perform their assessments (e.g., call into automated phone system) earlier than their usual time. Accordingly, another factor in trending index score determination (e.g., an additional term in any of Equations (1)-(5) or their variants described herein) may include the addition of a term generally correlating to the following difference: (average timestamp of previous N assessments—current assessment timestamp), where N assessments is some predetermined number (e.g., 5, 7, 10, etc.). Thus, generally, earlier assessments may result in a higher trending index score by virtue of their timestamp, and may, for example, help reduce the incidence of false positives or increase confidence levels of trending index score determination.

The trending index score can be used to trigger communication of an alert to a medical care provider and/or other contact, as described below. The alert may be triggered, for example, by comparing the index score to one or more thresholds. As described elsewhere herein, the threshold may be patient-specific, demographic-specific, etc. In an exemplary variation, an alert may be communicated if the trending index score is greater than 4.

Additionally or alternatively, patients may be ranked by their trending index scores (e.g., sorted based on trending index scores) where patients with higher trending index scores are considered higher risk of experiencing a respiratory or other clinical event relative to other patients, and thus may warrant medical attention or follow up. The ranking may be refreshed periodically (e.g., daily or every other day). As an illustrative example, among a group of 300 monitored patients assigned to a medical team, the trending index ranking may allow a medical team to know which 10-20 patients are at higher risk relative to the other 290 or 280 patients who are at lower risk on any given day.

In some variations, monitored patients may be triaged or sorted into tiered groups (e.g., high-risk, moderate-risk, low-risk, etc.) based on one or more of the above variations of assessing patient data. For example, patients may be sorted into an “alerts” or “red” list of patients, or into a “watchlist” or “yellow” list of patients corresponding to their likelihood of requiring medical attention. In some variations, patients with a higher trending index score (e.g., above a first predetermined threshold) may be sorted into an “alerts” group, and/or patients with moderate trending index scores may be sorted into a “watchlist” group. Additionally or alternatively, in some variations, patient ranking (e.g., based on trending index score) may additionally or alternatively be used to triage patients into tiered groups of patients. For example, a certain segment of ranked patients (e.g., five highest-ranking patients) may be sorted into an “alerts” or “watchlist” group of patients. Thresholds for tiering or grouping patients may be adjusted and/or confirmed by medical care practitioners as desired.

### Machine Learning Models

In some variations, various methods, systems, and apparatus, for predicting COPD flares or other respiratory conditions may utilize machine learning models such as neural networks. For example, a system may collect multimodal patient data (e.g., as described above), process the data into an encoded representation, make prediction on the data, and execute on the predictions. A neural network may be trained to process multimodal patient data using a recurrent neural network to predict and forecast COPD flaring as well as future patient biomarkers. Every day, new data is collected that improves and refines the predictive model.

### Input Mapping

Patient data including respiratory rate, heart rate, carbon dioxide retention, nocturnal cough, productive or non-productive coughing, and/or telemetric voice data may be recorded on a daily basis and sent to a server along with a recorded time stamp and patient ID. The sensory inputs may be measured using any of a variety of means, including but not limited to, an auto-dialer, an electric sensor, or self-reported question-answering (e.g, as described above). Single value readings, such as heart rate, respiratory rate, carbon-dioxide retention, and self-assessed responses may, for example, be recorded as single float values in the system. Discrete sequence readings, such as nightly productive or non-productive coughing, may be mapped to a sequence of floats. Continuous sequence readings, such as voice calls, heart recordings, and respiratory sounds, may be processed with a discrete wavelet transform. Principle Component Analysis or another suitable process may then used to reduce the dimensionality of the data and discover correlations between the features. Principle components can, for example, be accepted up to the point that 90% of the variance of the data is explained with the principle component mappings.

Each patient has a fixed size vector to represent the recorded values for an assessment (e.g., each day, for daily assessment). However, due to the nature of the data collection (e.g., forgetfulness in performing assessments, equipment failure, etc.), some patients may miss recording at least some data for an expected assessment. To compensate for missing data, in some implementations, the missing values, may be recorded as being the previous recorded value or, where that does not exist for the patient, as the baseline average for the risk and age category of the patient, which may be computed as an average for different age and COPD risk groups. In some implementations, the recurrent neural network model is trained to predict the next day's recorded values, so that in the event of missing data, the recurrent neural network can fill in the missing data with predictions.

### Structure of the Model

A recurrent neural network takes as input a sequence of patient data and outputs at each timestep the probability that a flare will occur within the next seven days. The recurrent neural network may take the form of a Long-Short-Term Memory (LSTM) network, a Gated Recurrent Unit (GRU),or any other architectural variation of the recurrent neural network. The architecture may include multiple deep layers of a stacked recurrent neural network, and may include attention modules to focus on previous time steps. At each timestep, the hidden state of the neural network outputs a probability that a flare occurs within the next seven days (or other suitable time interval).

### Training Process

The training data for training the model includes the patient sequence data as inputs and patient sequence data and flare events as labels. Data may, for example, be collected for more than 1000 patients over a period of at least a month to train the initial model. In an exemplary variation, on the day of a flare, the patient data for that day is labeled as a 2. For six days before each flare event, the time event for that day is labeled as a 1 indicating that a flare will occur within seven days. Days where no flare occurs in seven days are labeled as 0. However, specific labels may be adjusted appropriate for other variations.

In some variations, the recurrent neural network is trained using the backpropagation-through-time (BPTT) algorithm. The model iterates over a batch of patients whose entire sequence over the month or greater time recorded is trained in the model at once. A patient may have zero, one, or more than one flare events within a given sequence. For each timestep, the BPTT algorithm may back propagate gradients on a dual loss function that measures the correctness of predicting the flare event as well as the correctness of predicting the next day's patient data. The loss for predicting the flare event may be measured using the binary cross entropy function, and the loss for predicting the next day's patient data is predicted using the L2 loss against all fields of data where the next day's field is recorded. In some variations, where the next day's field is not recorded, that field is not included in the L2 loss.

The training process may be further supported by other techniques, including but not limited to, empirically testing different optimizers, empirically testing different hyper parameters including batch size, learning rate, decay rate, sizes of hidden units, batch normalization, and empirically testing different regularizations including but not limited to L1 and L2 regularizers, etc.

In some variations, the neural network may additionally or alternatively be trained in an online fashion. For example, every month after deployment, the model may be retrained with that month's new data and evaluated against the previous model on metrics including but not limited to accuracy of flare prediction and quality of next-day patient data predictions.

### Execution Phase

After a period of model training with an initial dataset, the model may be deployed in a server environment to predict daily the probability that a flare will occur within a week for a patient and the next day's patient data. A decision-making module may respond to the daily patient predictions and decide when to advise medical attention. When for two days in a row (or other suitable threshold) the model predicts the occurrence of a flare within the week (or other time interval) with greater than 50% probability, and the symptoms are predicted to worsen in the next day, the module may inform the patient and/or monitoring medical team to seek appropriate attention. In some variations, results of the prediction using the model may be used to group patients into tiered lists (e.g., “Alerts”, “Watchlist” etc.) as described above.

### Communicating Alert and Performing Medical Review

As shown in FIG. 10, an alert (e.g., a programmed alert) may be transmitted or otherwise communicated, such as based on predicted likelihood of an upcoming respiratory event (1050) or other characterization of patient risk and/or severity of medical condition. The alert may be communicated, for example, to one or more medical care providers 140. The alert may be received (1060) in any suitable manner. Generally, for example, the alert may be communicated through a messaging platform (e.g., push notifications on a mobile application, text message, etc.), push display options on a computing device (e.g., dedicated computer tablet, etc.), phone call, and/or fax machine. Additionally or alternatively, the alert may include displaying patients grouped into an “alerts” list and a “watchlist” (or other suitable patient tiering methodology) on a user interface. Any suitable combination of these communication methods may be used to notify alerts and prompt further medical review of one or more patients. For example, in addition to an electronic-based notification of a patient alert (e.g., as shown in any of the GUIs described below) or other electronic messaging platform, a HIPAA-compliant report for a patient associated with an alert may be faxed as a hard copy to the patient's medical team. In some variations, a combination of electronic and non-electronic methods increases redundancy of alert notifications, thereby reducing the possibility of medical care team overlooking an alert and/or reducing delay of a patient receiving medical review (such as if medical staff is not currently viewing the web portal or electronic messaging).

The medical care provider may perform a medical review of data and other information relating to the patient's medical treatment plan (1062) such as through a web portal, mobile application, or other suitable display interface (e.g., after logging into the system utilizing login credentials). The medical care provider may furthermore contact the patient via video, phone, text, or other message service as part of the remote evaluation. The provider may add notes to the patient's record, export files (e.g., for billing purposes), and/or perform other administrative actions.

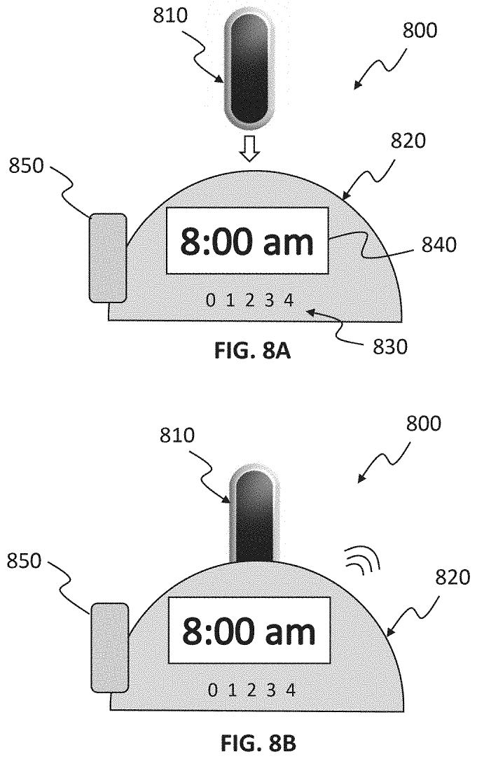

FIG. 11 illustrates an exemplary GUI 1100 through a web portal for a medical care provider to remotely view patient data. As shown in FIG. 11, the GUI 1100 may include an overview summary of the medical care provider's patients, including a summary of patient status (e.g., number of patients triggering alerts, requiring follow-up, or having no associated events or action items). Furthermore, GUI 1100 may include a medical care provider summary providing statistics such as a summary of remote monitoring success (e.g., 30-day hospital re-admission rate for the provider, versus a national average of the same).

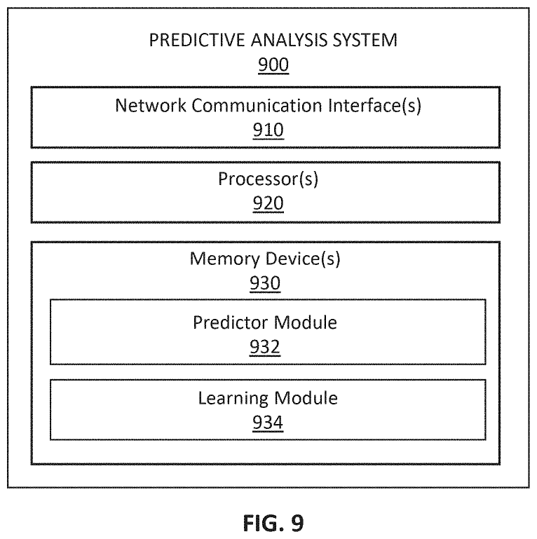

FIG. 12 illustrates another exemplary GUI 1200 through a web portal for a medical care provider to remotely view patient data. As shown in FIG. 12, the GUI 1200 may include a detailed view of specific patients and their status. GUI 1200 may include a triaged list of patients that places the highest at-risk patients at the top of the list. Each patient may be selectable in GUI 1200 to view additional information relating to that patient. GUI 1200 may, for example, identify patients whose most recent risk characterizations meet threshold levels, thereby allowing medical teams to more quickly focus their efforts and selectively view only the patients at higher risk. Additionally or alternatively, a notification (e.g., email or intra-software program message) may be provided to a medical care provider (e.g., physician, staff, etc.) that identifies one or more highest risk patients. Such a notification may include an embedded link to that patient's medical information for direct access to reported patient data.

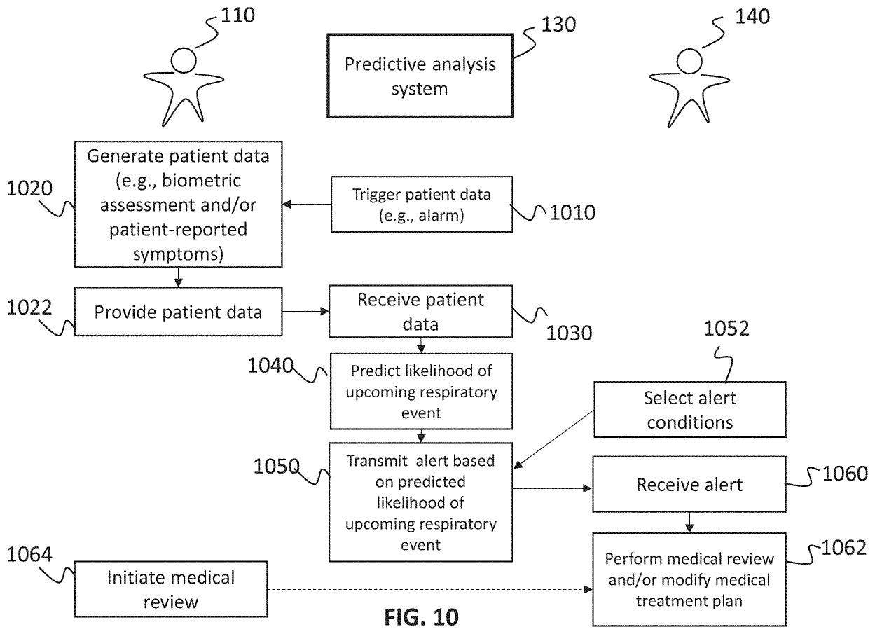

FIG. 13 illustrates another exemplary GUI 1300 through a web portal for a medical care provider to remotely view detailed patient data. As shown in FIG. 13, the GUI 1300 may display more detailed information relating to patient data as described above with reference to FIG. 12. GUI 1300 may, for example, display bodily metrics (e.g., respiratory rate, oxygen saturation, heart rate, breath sounds, changes in patient-reported symptoms), current medications, compliance with current medications and/or other data provided by the system.

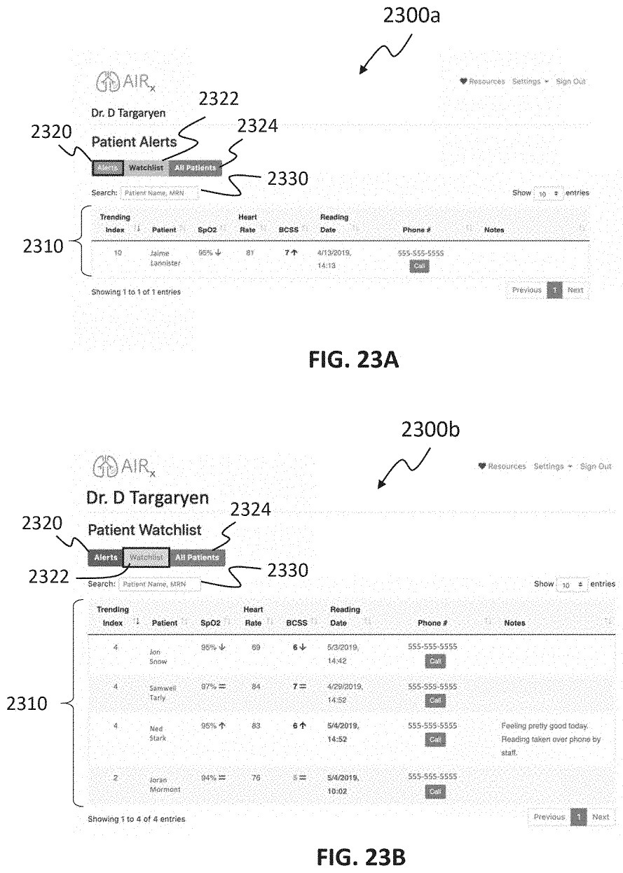

FIGS. 23A and 23B illustrate additional exemplary GUIs 2300a and 2300b for a medical care provider to remotely view triaged patients according to an “alerts” list (e.g., patients determined to be more likely to need immediate medical attention) as shown in GUI 2300a and a “watchlist” (e.g., patients who are determined to possibly need medical attention in the near future, but are less likely to need it immediately) as shown in GUI 2300b. For example, an “alerts” list of patients may be accessible by clicking button or tab 2320 labeled “Alerts”, while a “watchlist” of patients may be accessible by clicking button or tab 2322 labeled “Watchlist.” A list of all monitored patients may be accessible by clicking button or tab 2324 labeled “All Patients.” Furthermore, a list of one or more individual monitored patients may be accessible by entering patient information (e.g., name, medical record, etc.) into a search bar 2330. Patient information region 2310 may display information (e.g., trending index score, patient name, bodily metrics, contact information, notes, etc.) associated with selected or searched patients. As described above, the thresholds and other parameters for determining which patients are on an “alerts” or “watchlist” list of patients may be adjusted or confirmed by a medical care practitioner or other administrator.

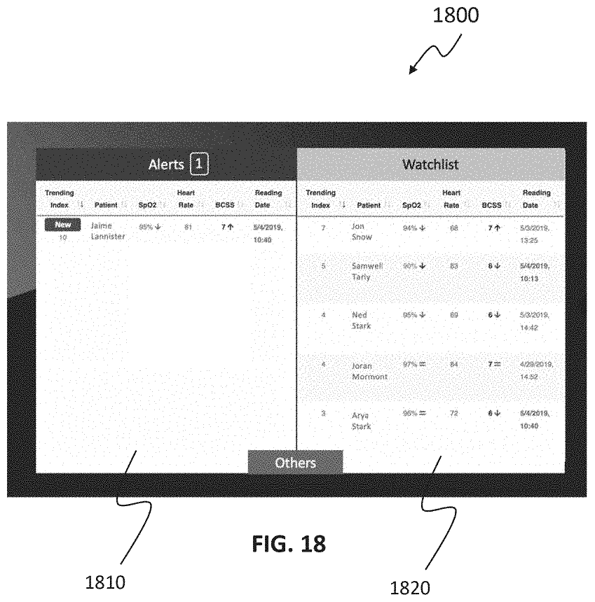

FIG. 18 illustrates another exemplary GUI 1800 for a medical care provider to remotely view triaged groupings of patients according to an “alerts” list 1810 including patients who are determined to be more likely to need medical attention and a “watchlist” 1820 including patients who are determined to possibly need medical attention in the near future. GUI 1800 may, for example, be displayed on a dedicated electronic device (e.g., tablet device, television screen, “smart” interactive whiteboard or other markerboard, etc.). Such a dedicated electronic may, for example, be placed in a centralized location (e.g., staff room) mounted on a wall, on a stand, or in another suitable easily-visible, preferably HIPAA-compliant manner. The electronic device may be interactive. For example, a user (e.g., medical staff) may provide a user input for selecting a patient listed with GUI 1800 (e.g., viewing patient data, confirming receipt and viewing of an alert, indicating whether the patient has been contacted, making notes on follow-up action items such as modifying treatment plan, etc.). In some variations, GUI 1800 may enable tracking of whether an alert has been viewed and/or addressed. The information displayed on GUI 1800 may be updated continuously in real-time or periodically (e.g., every hour or half-hour). In some variations, GUI 1800 on a dedicated electronic device may help reduce alert or “portal” fatigue, and help focus attention on those patients who are more likely to need medical attention. It should be understood that the format of GUI 1800 may additionally or alternatively be implemented on a personal computer (e.g., associated with an individual medical care practitioner) or mobile computing device, or other electronic device.

### Modifying Medical Treatment Plan

After remotely reviewing the patient information, the medical care provider may modify one or more aspects of the patient's medical treatment plan, such as modifying medications. For example, the medical care provider may modify the patient's inhaler, steroids, antibiotics, etc. In some variations, modifying a medical treatment plan may include changing the dose or frequency of one or more of the following, alone or in combination: short-acting beta agonists, short-acting muscarinic antagonists, long-acting beta agonists, long-acting muscarinic antagonists, inhaled corticosteroids, antibiotics, supplemental oxygen, nebulizer treatment, and pulmonary rehabilitation. Alternatively, a medical treatment plan may be modified automatically in response to the alert, without medical review. After this review and/or modification of the medical treatment plan, the medical care practitioner may perform follow-up review and/or additional modification of the medical treatment plan depending on the patient's response to the modified treatment.

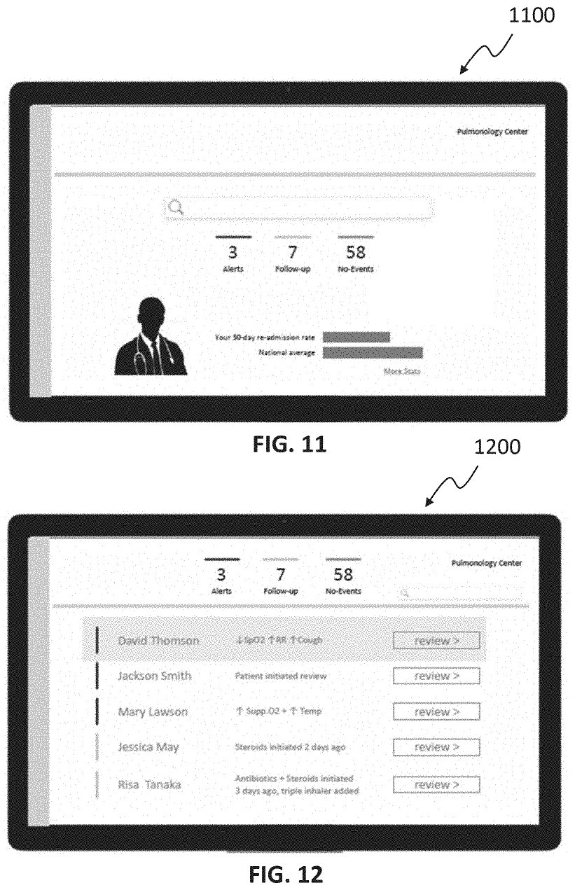

FIG. 14 illustrates an exemplary GUI 1400 through a web portal for a medical care provider to view individual patient data (and trends thereof) and/or remotely modify the medical plan.

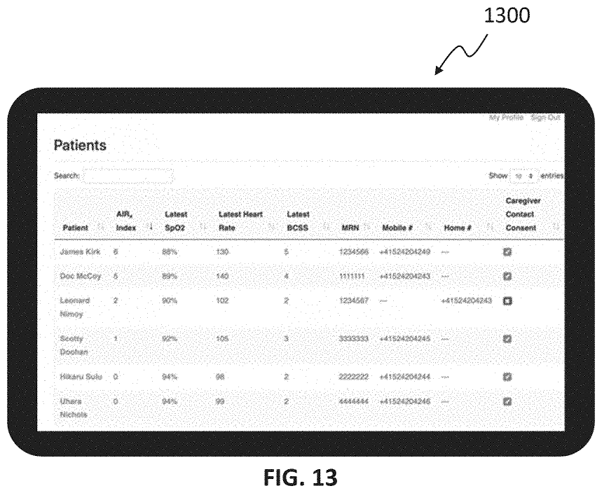

FIG. 15 illustrates an exemplary GUI 1500 through a web portal for a medical care provider to select or enter aspects of the patient's medical plan. The medical care provider may choose to communicate with the patient (e.g., through video, phone, text, a note, or other mode) through the GUI 1500. In some variations, such communications (e.g., audio and/or text-based notes) may be automatically imported to the patient's electronic medical record.

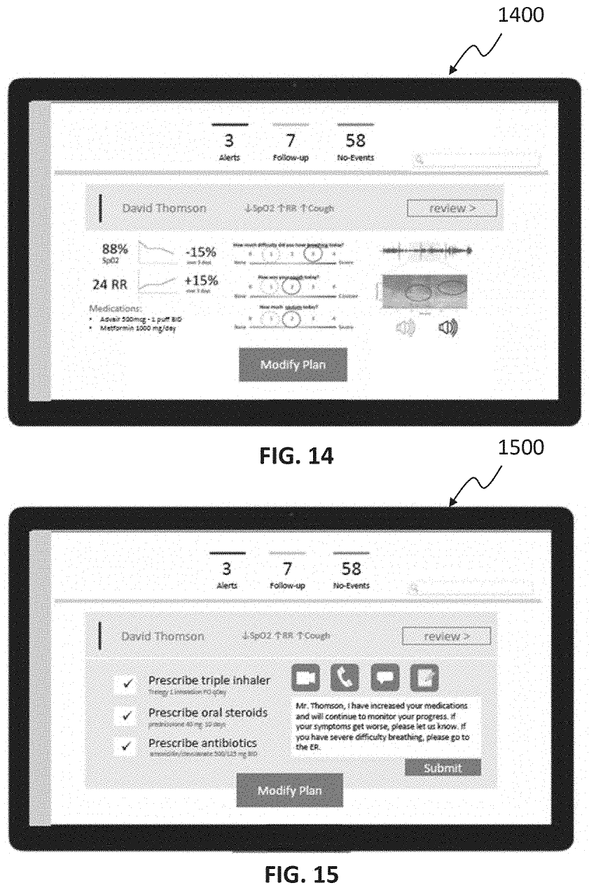

FIG. 16 illustrates another exemplary GUI 1600 through a web portal for a medical care provider to view a confirmation of changes made to a medical treatment plan. The system may automatically update the patient's electronic medical record accordingly. A notification may also be provided to confirm that monetary credit is added to the account of the medical care provider based on the actions.

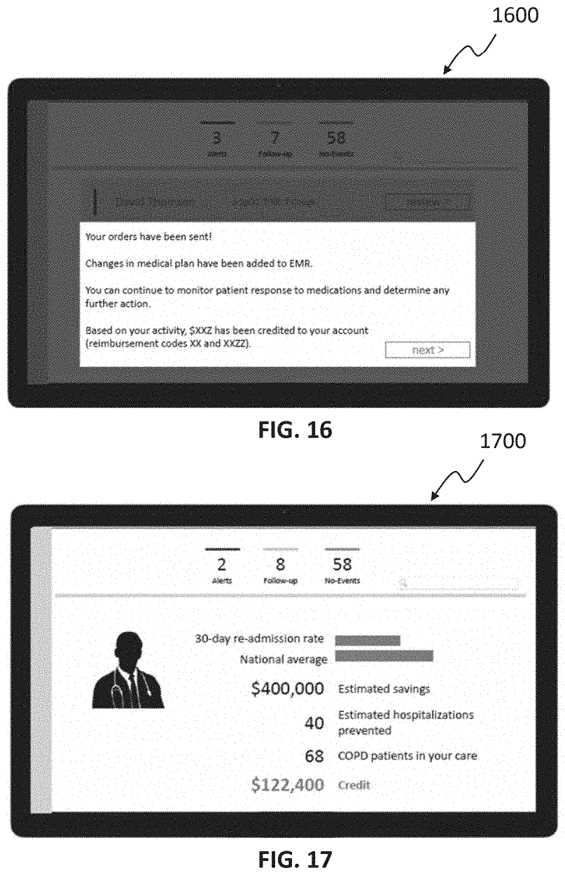

FIG. 17 illustrates an exemplary GUI 1700 through a web portal whereby a medical care provider may view data on his or her provider performance, (e.g., 30-day readmission rate) compared against other providers (e.g., against national average). Additional statistics such as total estimated savings, estimated number of hospitalizations prevents, and number of patients in his or her care, may also be provided in GUI 1700.

### EXAMPLE 1

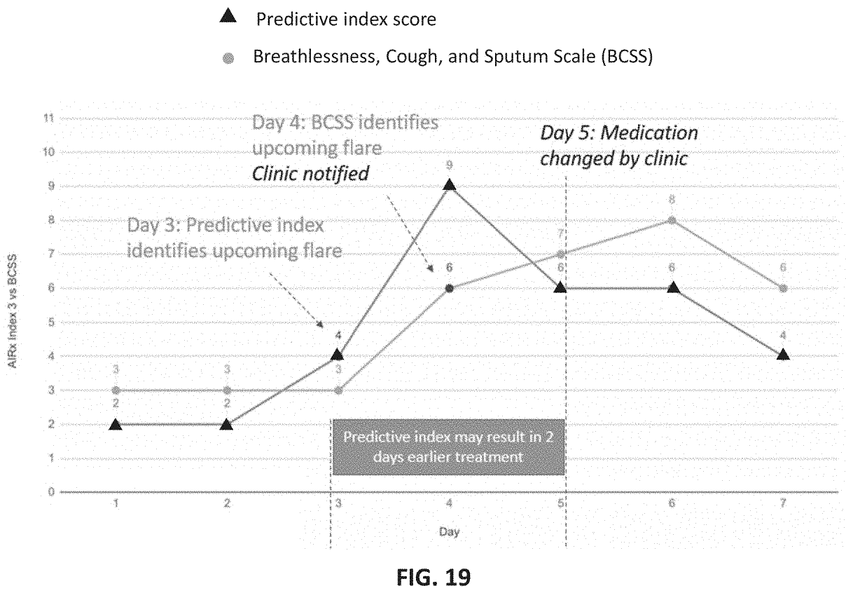

FIG. 19 illustrates trends in a bodily metric (BCSS score) and predictive index score for a remotely monitored patient who experienced a COPD flare. Specifically, BCSS score for the patient was found to increase over a monitoring period, peaking on Day 4 when a medical care provider was notified. The BCSS score value of 6 on Day 4 was used to identify an upcoming COPD flare. On Day 5, the patient's medication was changed by the clinic to address the COPD flare. Following the change in medication, the patient's BCSS score gradually began to drop.

Overlaid over the BCSS score trend is a graph of a predictive index score for the patient, generated according to Equation 3 above. On Day 3, the predictive index score increased to 4, which could have been used to trigger an alert for communication to the clinic as described above. In other words, the predictive index score (based on the oxygen saturation, heart rate, and BCSS scores as remotely provided during monitoring) may have been used to provide treatment to the patient 2 days earlier than otherwise. Accordingly, the systems and methods described herein may be used to successfully remotely monitor patients, identify upcoming respiratory events, and facilitate more responsive and effective medical treatment to avoid or mitigate such upcoming respiratory events.

### EXAMPLE 2

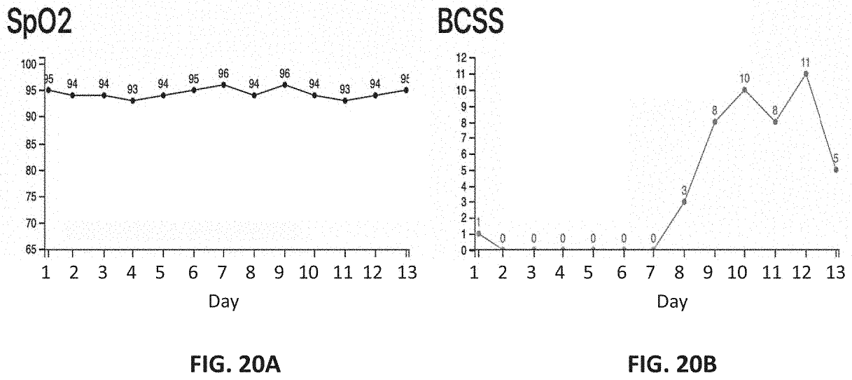

FIGS. 20A and 20B illustrate trends in SpO₂ and BCSS score, respectively, for a remotely monitored patient. Over the course of remote monitoring as described above, a drastic rise of BCSS score over 48 hours (days 8 and 9) was detected. In response to the remote monitoring and detection of the significant rate of increase in BCSS score, the patient's clinic prescribed medication. As shown in FIG. 20B, remote monitoring of the patient's bodily metrics continued during the medication treatment, and captured a general recovery of the patient's symptoms (days 11-13). Accordingly, the systems and methods described herein may be used by clinics to successfully remotely monitor conditions of patients, adjust treatment, and then follow-up to characterize the level of success with treatment (e.g., remotely monitor whether and how successfully the patients respond to treatment) and/or determine whether the patients require further intervention.

### EXAMPLE 3

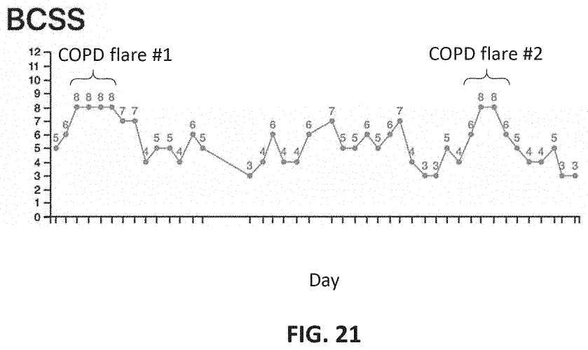

FIG. 21 illustrates trends in BCSS score for a remotely monitored patient having a relatively high or elevated baseline BCSS score. Over the course of remote monitoring as described above, the patient experienced two COPD flares (“COPD flare #1” and “COPD flare #2”) reflected by an elevated BCSS score of 8 within 42 days of being monitored. Following each COPD flare, the patient's clinic remotely prescribed medication changes (steroids) to reduce the patient's symptoms and thereby treat the patient's COPD exacerbations at home, instead of in the emergency room. The general decline in BCSS score following each COPD flare and medication change strongly suggests a successful response to treatment. Accordingly, the systems and methods described herein may be used by clinics to successfully remotely monitor conditions of patients, adjust treatment, and then follow-up to characterize the level of success with treatment (e.g., remotely monitor whether and how successfully the patients respond to treatment) and/or determine whether the patients require further intervention.

### EXAMPLE 4

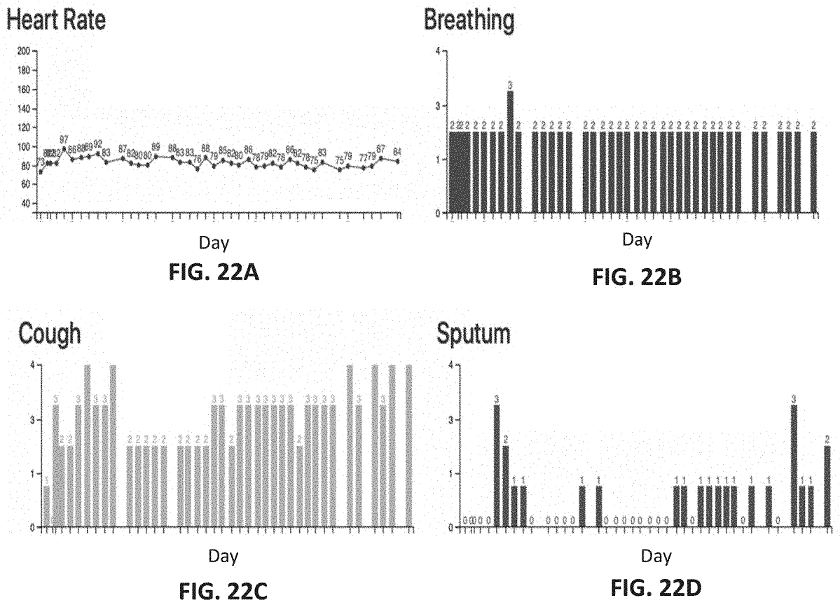

FIGS. 22A-22D illustrate trends in various bodily metrics for a remotely monitored patient, including heart rate (FIG. 22A), breathlessness severity (FIG. 22B), cough severity (FIG. 22C), and sputum severity (FIG. 22D). When the breathlessness severity, cough severity, and sputum severity were initially combined into BCSS score for monitoring, the resulting BCSS score was relatively high, which ordinarily would suggest a COPD exacerbation for the patient. However, upon further investigation in which the components of BCSS score were individually assessed, it was determined that the patient had disproportionately severe cough (maximum or near-maximum levels) relative to breathlessness and sputum severity. Based on this disproportionately severe cough, the patient's clinic proceeded to perform additional, specialized investigations (e.g., medical work-up to investigate for lung malignancy) to determine other possible diagnoses. Accordingly, the systems and methods described herein may additionally or alternatively be used by clinics to monitor specific symptoms for triggering a range of medical diagnostic investigations in order to determine suitable medical treatment for a patient.

### EXAMPLE 5

In another example, a method for monitoring a respiratory condition includes monitoring asthma. In this example, the method may include receiving one or more bodily metrics including peak expiratory flow rate (PEFR), such as over an automated phone system as described above. Specifically, a patient can measure their PEFR during an assessment using an issued PEFR meter, then recite or otherwise enter PEFR values over the automated phone system, which may then be compared to patient baseline data (e.g., best PEFR) to assess the patient. For example, a patient may be considered in a “normal” tier if current PEFR is about 80% or more of the patient's baseline best PEFR, a yellow or “watchlist” tier if current PEFR is between about 50%-79% of the patient's baseline best PEFR, and a red or “alerts” tier if current PEFR is less than about 50% of the patient's baseline best PEFR.

Additionally, during an assessment a patient may recite, enter, or otherwise provide symptoms such as cough, wheezing, chest tightness, shortness of breath, etc. which may be associated with a symptom report score (e.g., 1-3, in increasing severity). For example, a symptom report score of 1 may be associated with no coughing, wheezing, chest tightness, or shortness of breath during the day or night, and an assessment that the patient can do usual activities. A symptom report score of 2 may be associated with coughing, wheezing, chest tightness, or shortness of breath, or waking at night due to asthma, and an assessment that the patient can participate in a reduced amount of activities. A symptom report score of 3 may be associated with extreme shortness of breath, or an assessment that quick-relief medications do not help or that the patient amount participate in usual activities, or symptoms are the same or get worse after 24 hours. Accordingly, such bodily metrics and symptoms may be used to characterize risk (e.g., tier) of the patient having an asthma event.

### Enumerated Embodiments

Embodiment A1. A method for remotely monitoring chronic respiratory conditions of a plurality of patients, the method comprising:
at one or more processors:
receiving a plurality of bodily metrics associated with each of the plurality of patients over an automated phone system;
characterizing the severity of the respiratory condition for each patient based on the plurality of bodily metrics associated with the patient; and
transmitting a programmed alert to a medical care provider for at least one patient having a respiratory condition characterized as having a threshold level of severity.

Embodiment A2. The method of embodiment A1, wherein receiving the plurality of bodily metrics comprises prompting at least one patient during a phone call established with the automated phone system to provide at least a portion of the plurality of bodily metrics.

Embodiment A3. The method of embodiment A1 or A2, wherein receiving the plurality of bodily metrics comprises receiving and recording one or more bodily metrics recited by the at least one patient over the automated phone system, wherein the method further comprises performing speech-to-text transcription of the recording.

Embodiment A4. The method of any one of embodiments A1-A3, wherein receiving the plurality of bodily metrics comprises receiving one or more bodily metrics entered through a telephone keypad interface.

Embodiment A5. The method of any one of embodiments A1-A4, further comprising receiving the phone call through the automated phone system from a patient phone number associated with the at least one patient, and masking the patient phone number with a second phone number to de-identify the at least one patient.

Embodiment A6. The method of any one of embodiments A1-A5, wherein characterizing the severity of the respiratory condition comprises assessing at least a portion of the received bodily metrics substantially in real-time during the phone call.

Embodiment A7. The method of any one of embodiments A1-A6, further comprising assessing the received bodily metrics, dynamically generating one or more supplemental prompts based on the assessment of at least one received bodily metric, and prompting at least one patient with the one or more supplemental prompts over the automated phone system to provide supplemental patient data in response to the one or more supplemental prompts.

Embodiment A8. The method of any one of embodiments A1-A7, wherein the one or more supplemental prompts is generated based on at least one received bodily metric satisfying at least one predetermined dynamic threshold, wherein the at least one predetermined dynamic threshold is customizable with respect to a medical care provider, the at least one patient, or both.

Embodiment A9. The method of any one of embodiments A1-A8, wherein the automated phone system is configured with a programmable communications platform.

Embodiment A10. The method of any one of embodiments A1-A9, wherein the programmable communications platform comprises at least one of a programmable voice service and a programmable text service.

Embodiment All. The method of any one of embodiments A1-A10, wherein characterizing the severity of the respiratory condition for each patient comprises comparing at least a first bodily metric to a first predetermined threshold.

Embodiment A12. The method of any one of embodiments A1-A11, wherein the first predetermined threshold is customizable with respect to a medical care provider, one or more patients, or both.

Embodiment A13. The method of any one of embodiments A1-A12, wherein the plurality of bodily metrics is received at least once daily over a monitoring period of two or more days.

Embodiment A14. The method of any one of embodiments A1-A13, further comprising contacting at least one of a patient and a patient-designated contact person in response to failing to receive the plurality of bodily metrics for the patient within a predetermined time window.

Embodiment A15. The method of any one of embodiments A1-A14, wherein the medical care provider is associated with a virtual clinic.

Embodiment A16. The method of any one of embodiments A1-A15, further comprising establishing a patient-medical care provider relationship for at least one of the plurality of patients through an asynchronous store and forward transmission of medical information associated with the at least one patient, from an originating site to the medical care provider not at the originating site, and without a real-time presence of the patient.

Embodiment A17. The method of any one of embodiments A1-A16, further comprising verifying an identity of the at least one patient through a non-documentary identity verification process.

Embodiment A18. The method of any one of embodiments A1-A17, further comprising receiving an intake questionnaire to be reviewed by the medical care provider.

Embodiment A19. The method of any one of embodiments A1-A18, wherein the medical care provider is a first medical care provider comprising an auxiliary staff member working under supervision of a second medical care provider, wherein the first medical care provider and the second medical care provider are working in different physical locations, at different times, or both.

Embodiment A20. The method of any one of embodiments A1-A19, wherein the medical care provider provides designated medical care coverage during a covering period in the absence of a primary medical care provider associated with at least one patient.

Embodiment A21. The method of any one of embodiments A1-A20, wherein the plurality of bodily metrics comprises at least one of oxygen saturation, heart rate, breathlessness severity, cough severity, and mucus severity for the at least one patient.

Embodiment A22. The method of any one of embodiments A1-A21, wherein receiving the plurality of bodily metrics comprises receiving and recording a phone call during which one or more bodily metrics is recited over an automated phone system, wherein the plurality of bodily metrics further comprises respiratory rate, and wherein the method comprises measuring respiratory rate at least in part by counting patient breaths detected in the recording.

Embodiment A23. The method of any one of embodiments A1-A22, wherein the plurality of bodily metrics further comprises a quantification of home supplemental oxygen used by the at least one patient.

Embodiment A24. The method of any one of embodiments A1-A23, further comprising generating an index score for each patient, wherein the index score corresponds to the characterized severity of the patient, and further comprising sorting the plurality of patients according to the index scores for the plurality of patients.

Embodiment A25. The method of any one of embodiments A1-A24, wherein sorting the plurality of patients comprises grouping each of the plurality of patients into one of a low-risk category, a medium-risk category, and a high-risk category.

Embodiment A26. The method of any one of embodiments A1-A25, wherein characterizing the severity of the respiratory condition comprises deriving a first points value for each bodily metric for at least one patient, and wherein the index score for the at least one patient based at least in part on the first points values for the plurality of bodily metrics.

Embodiment A27. The method of any one of embodiments A1-A26, wherein receiving a plurality of bodily metrics is repeated for the at least one patient over a monitoring period, and wherein characterizing the severity of the respiratory condition comprises:
deriving a second points value of at least one bodily metric for the at least one patient based on the relative change of the at least one bodily metric during the monitoring period,
wherein the index score based at least in part on the first points values for the plurality of bodily metrics and the second points value of at least one bodily metric.

Embodiment A28. The method of any one of embodiments A1-A27, further comprising enabling the medical care provider to adjust a medical treatment for at least one patient in response to the transmitted programmed alert.

Embodiment A29. The method of any one of embodiments A1-A28, further comprising providing a reminder to the at least one patient over the automated phone system to conform to prescribed medical treatment, or inviting the at least one patient to ask a question to the medical care provider, or both.

Embodiment A30. The method of any one of embodiments A1-A29, wherein the automated phone system is configured to facilitate interactive communication between the medical care provider and at least one patient.

Embodiment B1. A method for remotely monitoring chronic medical conditions of a plurality of patients, the method comprising:

at one or more processors:
receiving a plurality of bodily metrics associated with each of the plurality of patients over one or more remote communication links;
characterizing the severity of the medical condition for each patient based on the plurality of bodily metrics associated with the patient; and
transmitting a programmed alert to a medical care provider for at least one patient having a medical condition characterized as having a threshold level of severity.

Embodiment B2. The method of embodiment B1, wherein receiving a plurality of bodily metrics comprises receiving a plurality of bodily metrics associated with each of a plurality of patients over an automated phone system.

Embodiment B3. The method of embodiment B1 or B2, wherein receiving the plurality of bodily metrics comprises prompting at least one patient during a phone call established with the automated phone system to provide at least a portion of the plurality of bodily metrics.

Embodiment B4. The method of any one of embodiments B1-B3, wherein receiving the plurality of bodily metrics comprises receiving and recording one or more bodily metrics recited by the at least one patient over the automated phone system, wherein the method further comprises performing speech-to-text transcription of the recording.

Embodiment B5. The method of any one of embodiments B1-B4, wherein receiving the plurality of bodily metrics comprises receiving one or more bodily metrics entered through a telephone keypad interface.

Embodiment B6. The method of any one of embodiments B1-B5, further comprising receiving the phone call through the automated phone system from a patient phone number associated with the at least one patient, and masking the patient phone number with a second phone number to de-identify the at least one patient.

Embodiment B7. The method of any one of embodiments B1-B6, wherein characterizing the severity of the medical condition comprises assessing at least a portion of the received bodily metrics substantially in real-time during the phone call.

Embodiment B8. The method of any one of embodiments B1-B7, further comprising assessing the received bodily metrics, dynamically generating one or more supplemental prompts based on the assessment of at least one received bodily metric, and prompting at least one patient with the one or more supplemental prompts over the automated phone system to provide supplemental patient data in response to the one or more supplemental prompts.

Embodiment B9. The method of any one of embodiments B1-B8, wherein the one or more supplemental prompts is generated based on at least one received bodily metric satisfying at least one predetermined dynamic threshold, wherein the at least one predetermined dynamic threshold is customizable with respect to a medical care provider, the at least one patient, or both.

Embodiment B10. The method of any one of embodiments B1-B9, wherein the automated phone system is configured with a programmable communications platform.

Embodiment B11. The method of any one of embodiments B1-B10, wherein the programmable communications platform comprises at least one of a programmable voice service and a programmable text service.

Embodiment B12. The method of any one of embodiments B1-B11, wherein characterizing the severity of the medical condition for each patient comprises comparing at least a first bodily metric to a first predetermined threshold.

Embodiment B13. The method of any one of embodiments B1-B12, wherein the first predetermined threshold is customizable with B12 to a medical care provider, one or more patients, or both.

Embodiment B14. The method of any one of embodiments B1-B13, further comprising generating an index score for each patient, wherein the index score corresponds to the characterized severity of the patient, and further comprising sorting the plurality of patients according to the index scores for the plurality of patients.

Embodiment B15. The method of any one of embodiments B1-B14, wherein sorting the plurality of patients comprises grouping each of the plurality of patients into one of a low-risk category, a medium-risk category, and a high-risk category.

Embodiment B16. The method of any one of embodiments B1-B15, wherein the plurality of bodily metrics is received at least once daily over a monitoring period of two or more days.

Embodiment B17. The method of any one of embodiments B1-B16, further comprising contacting at least one of a patient and a patient-designated contact person in response to failing to receive the plurality of bodily metrics for the patient within a predetermined time window.

Embodiment B18. The method of any one of embodiments B1-B17, wherein the medical care provider is associated with a virtual clinic.

Embodiment B19. The method of any one of embodiments B1-B18, further comprising establishing a patient-medical care provider relationship for at least one of the plurality of patients through an asynchronous store and forward transmission of medical information associated with the at least one patient, from an originating site to the medical care provider not at the originating site, and without a real-time presence of the patient.

Embodiment B20. The method of any one of embodiments B1-B19, further comprising verifying an identity of the at least one patient through a non-documentary identity verification process.

Embodiment B21. The method of any one of embodiments B1-B20, further comprising receiving an intake questionnaire to be reviewed by the medical care provider.

Embodiment B22. The method of any one of embodiments B1-B21, wherein the medical care provider is a first medical care provider comprising an auxiliary staff member working under supervision of a second medical care provider, wherein the first medical care provider and the second medical care provider are working in different physical locations, at different times, or both.

Embodiment B23. The method of any one of embodiments B1-B22, wherein the medical care provider provides designated medical care coverage during a covering period in the absence of a primary medical care provider associated with at least one patient.

Embodiment B24. The method of any one of embodiments B1-B23, wherein at least one of the chronic medical conditions is a chronic respiratory condition, and wherein the plurality of bodily metrics comprises at least one of oxygen saturation, heart rate, breathlessness severity, cough severity, and mucus severity for the at least one patient.

Embodiment B25. The method of any one of embodiments B1-B24, wherein receiving the plurality of bodily metrics comprises receiving and recording a phone call during which one or more bodily metrics is recited over an automated phone system, wherein the plurality of bodily metrics further comprises respiratory rate, and wherein the method comprises measuring respiratory rate at least in part by counting patient breaths detected in the recording.

Embodiment B26. The method of any one of embodiments B1-B25, wherein the plurality of bodily metrics further comprises a quantification of home supplemental oxygen used by the at least one patient.

Embodiment B27. The method of any one of embodiments B1-B26, wherein characterizing the severity of the medical condition comprises deriving a first points value for each bodily metric for at least one patient, and generating an index score for the at least one patient based at least in part on the first points values for the plurality of bodily metrics.

Embodiment B28. The method of any one of embodiments B1-B27, wherein receiving a plurality of bodily metrics is repeated for the at least one patient over a monitoring period, and wherein characterizing the severity of the respiratory condition comprises: deriving a second points value of at least one bodily metric for the at least one patient based on the relative change of the at least one bodily metric during the monitoring period; and generating an index score based at least in part on the first points values for the plurality of bodily metrics and the second points value of at least one bodily metric.

Embodiment B29. The method of any one of embodiments B1-B28, further comprising enabling the medical care provider to adjust a medical treatment for at least one patient in response to the transmitted programmed alert.

Embodiment B30. The method of any one of embodiments B1-B29, further comprising providing a reminder to the at least one patient over the one or more remote communication links to conform to prescribed medical treatment, or inviting the at least one patient to ask a question to the medical care provider, or both.

Embodiment B31. The method of any one of embodiments B1-B30, wherein the automated phone system is configured to facilitate interactive communication between the medical care provider and at least one patient.

Embodiment C1. A method for managing a chronic respiratory condition of a patient, the method comprising:
at one or more processors:
receiving a plurality of bodily metrics over a remote communication link provided by a patient over an automated phone system; and
characterizing the risk of an upcoming clinical event for the patient based on the received bodily metrics.

Embodiment C2. The method of embodiment C1, further comprising prompting a designated contact in response to the patient failing to provide at least one of the plurality of bodily metrics.

Embodiment C3. The method of embodiment C1 or C2, wherein the plurality of bodily metrics is received at least once daily over a monitoring period of two or more days.

Embodiment C4. The method of any one of embodiments C1-C3, wherein the plurality of bodily metrics comprise at least one of oxygen saturation, heart rate, breathlessness severity, cough severity, and mucus severity.

Embodiment C5. The method of any one of embodiments C1-C4, wherein the plurality of bodily metrics further comprises at least one of vocal quality, respiratory rate, and respiratory sounds.

Embodiment C6. The method of any one of embodiments C1-C5, further comprising recording and processing a reference speech excerpt recited by the patient.

Embodiment C7. The method of any one of embodiments C1-C6, further comprising verifying at least one of the received plurality of bodily metrics in response to the at least one bodily metric being outside of a predetermined range.

Embodiment C8. The method of any one of embodiments C1-C7, wherein verifying the at least one received bodily metric comprises prompting a manual review of the at least one received bodily metric.

Embodiment D1. A method for managing a chronic respiratory condition of a patient, the method comprising:
remotely monitoring a patient by receiving patient status assessments via a remote communication link, wherein the patient status assessments comprise a plurality of bodily metrics associated with the respiratory condition;
characterizing risk of an upcoming clinical event for the patient based on the patient status assessments; and
adjusting a medical treatment for the patient in response to the characterized risk of an upcoming clinical event.

Embodiment D2. The method of embodiment D1, wherein the patient has been diagnosed with chronic obstructive pulmonary disease (COPD).

Embodiment D3. The method of embodiment D1 or D2, wherein the patient has been previously hospitalized for COPD.

Embodiment D4. The method of any one of embodiments D1-D3, wherein the patient has not been previously hospitalized for COPD.

Embodiment D5. The method of any one of embodiments D1-D4, wherein adjusting a medical treatment for the patient comprises modifying dose or frequency of one or more of the following: short-acting beta agonists, short-acting muscarinic antagonists, long-acting beta agonists, long-acting muscarinic antagonists, inhaled corticosteroids, antibiotics, supplemental oxygen, nebulizer treatment, and pulmonary rehabilitation.

Embodiment El. A system for remotely managing a chronic respiratory condition of a patient, the system comprising:
a measurement device comprising one or more sensors for measuring one or more bodily metrics of the patient, wherein the one or more sensors comprise a pulse oximeter for measuring at least one of oxygen saturation and heart rate; and
a docking station configured to removably receive the measurement device, wherein at least one of the measurement device and the docking station comprises a wireless communication system configured to transmit the one or more bodily metrics of the patient.

Embodiment E2. The system of embodiment E1, wherein the measurement device is a wearable device.

Embodiment E3. The system of embodiment E1 or E2, wherein at least one of the measurement device and the docking station further comprises a microphone.

Embodiment E4. The system of any one of embodiments E1-E3, wherein at least one of the measurement device and the docking station further comprises at least one of an audible notification and a visual notification.

Embodiment E5. The system of any one of embodiments E1-E4, wherein the docking station comprises a power supply to recharge a power source of the measurement device.

Embodiment F1. A wearable device for remotely managing a chronic respiratory condition of a patient, the system comprising:
a housing;
one or more sensors for measuring one or more bodily metrics of the patient, wherein the one or more sensors comprise a pulse oximeter for measuring at least one of oxygen saturation and heart rate; and
a microphone for recording at least one of respiratory and vocal sounds of the patient.

Embodiment F2. The wearable device of embodiment F1, wherein the housing comprises a pendant.

Embodiment F3. The wearable device of embodiment F1 or F2, wherein the pendant comprises a finger-receiving surface.

Embodiment F4. The wearable device of any one of embodiments F1-F3, wherein the housing comprises a patch.

Embodiment F5. The wearable device of any one of embodiments F1-F4, further comprising a wireless communication system configured to transmit the one or more bodily metrics of the patient.

Embodiment F6. The wearable device of any one of embodiments F1-F5, further comprising an alarm system configured to provide at least one of an audible notification and visual notification to remind the user to perform an assessment with the one or more sensors.

Embodiment G1. A system for remotely managing a chronic respiratory condition of a patient, the system comprising:
a housing comprising a receptacle configured to removably receive a measurement device having one or more sensors for measuring one or more bodily metrics of the patient;
a storage device configured to receive and store bodily metric data from the one or more sensors; and
a wireless communication module configured to transmit the stored bodily metric data.

Embodiment G2. The system of embodiment G1, further comprising a microphone.

Embodiment G3. The system of embodiment G1 or G2, further comprising a power supply to recharge a power source of the measurement device.

Embodiment G4. The system of any one of embodiments G1-G3, further comprising an alarm system configured to provide at least one of an audible notification and visual notification to remind the user to perform an assessment with the one or more sensors.

The foregoing descriptions, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1. A method for remotely monitoring chronic respiratory conditions of a plurality of patients, the method comprising:
at one or more processors:
receiving a plurality of bodily metrics associated with each of the plurality of patients over an automated phone system;
characterizing the severity of the respiratory condition for each patient based on the plurality of bodily metrics associated with the patient; and
transmitting a programmed alert to a medical care provider for at least one patient having a respiratory condition characterized as having a threshold level of severity.

2. The method of claim 1, wherein receiving the plurality of bodily metrics comprises prompting at least one patient during a phone call established with the automated phone system to provide at least a portion of the plurality of bodily metrics.

3. The method of claim 2, wherein receiving the plurality of bodily metrics comprises receiving and recording one or more bodily metrics recited by the at least one patient over the automated phone system, wherein the method further comprises performing speech-to-text transcription of the recording.

4. The method of claim 2, wherein receiving the plurality of bodily metrics comprises receiving one or more bodily metrics entered through a telephone keypad interface.

5. The method of claim 2, further comprising receiving the phone call through the automated phone system from a patient phone number associated with the at least one patient, and masking the patient phone number with a second phone number to de-identify the at least one patient.

6. The method of claim 2, wherein characterizing the severity of the respiratory condition comprises assessing at least a portion of the received bodily metrics substantially in real-time during the phone call.

7. The method of claim 1, further comprising assessing the received bodily metrics, dynamically generating one or more supplemental prompts based on the assessment of at least one received bodily metric, and prompting at least one patient with the one or more supplemental prompts over the automated phone system to provide supplemental patient data in response to the one or more supplemental prompts.

8. The method of claim 7, wherein the one or more supplemental prompts is generated based on at least one received bodily metric satisfying at least one predetermined dynamic threshold, wherein the at least one predetermined dynamic threshold is customizable with respect to a medical care provider, the at least one patient, or both.

9. The method of claim 1, wherein the automated phone system is configured with a programmable communications platform.

10. The method of claim 9, wherein the programmable communications platform comprises at least one of a programmable voice service and a programmable text service.

11. The method of claim 1, wherein characterizing the severity of the respiratory condition for each patient comprises comparing at least a first bodily metric to a first predetermined threshold.

12. The method of claim 11, wherein the first predetermined threshold is customizable with respect to a medical care provider, one or more patients, or both.

13. The method of claim 1, wherein the plurality of bodily metrics is received at least once daily over a monitoring period of two or more days.

14. The method of claim 13, further comprising contacting at least one of a patient and a patient-designated contact person in response to failing to receive the plurality of bodily metrics for the patient within a predetermined time window.

15. The method of claim 1, wherein the medical care provider is associated with a virtual clinic.

16. The method of claim 15, further comprising establishing a patient-medical care provider relationship for at least one of the plurality of patients through an asynchronous store and forward transmission of medical information associated with the at least one patient, from an originating site to the medical care provider not at the originating site, and without a real-time presence of the patient.

17. The method of claim 16, further comprising verifying an identity of the at least one patient through a non-documentary identity verification process.

18. The method of claim 16, further comprising receiving an intake questionnaire to be reviewed by the medical care provider.

19. The method of claim 15, wherein the medical care provider is a first medical care provider comprising an auxiliary staff member working under supervision of a second medical care provider, wherein the first medical care provider and the second medical care provider are working in different physical locations, at different times, or both.

20. The method of claim 15, wherein the medical care provider provides designated medical care coverage during a covering period in the absence of a primary medical care provider associated with at least one patient.

21. The method of claim 1, wherein the plurality of bodily metrics comprises at least one of oxygen saturation, heart rate, breathlessness severity, cough severity, and mucus severity for the at least one patient.

22. The method of claim 21, wherein receiving the plurality of bodily metrics comprises receiving and recording a phone call during which one or more bodily metrics is recited over an automated phone system, wherein the plurality of bodily metrics further comprises respiratory rate, and wherein the method comprises measuring respiratory rate at least in part by counting patient breaths detected in the recording.

23. The method of claim 21, wherein the plurality of bodily metrics further comprises a quantification of home supplemental oxygen used by the at least one patient.

24. The method of claim 1, further comprising generating an index score for each patient, wherein the index score corresponds to the characterized severity of the patient, and further comprising sorting the plurality of patients according to the index scores for the plurality of patients.

25. The method of claim 24, wherein sorting the plurality of patients comprises grouping each of the plurality of patients into one of a low-risk category, a medium-risk category, and a high-risk category.

26. The method of claim 24, wherein characterizing the severity of the respiratory condition comprises deriving a first points value for each bodily metric for at least one patient, and wherein the index score for the at least one patient based at least in part on the first points values for the plurality of bodily metrics.

27. The method of claim 24, wherein receiving a plurality of bodily metrics is repeated for the at least one patient over a monitoring period, and wherein characterizing the severity of the respiratory condition comprises:
deriving a second points value of at least one bodily metric for the at least one patient based on the relative change of the at least one bodily metric during the monitoring period,
wherein the index score based at least in part on the first points values for the plurality of bodily metrics and the second points value of at least one bodily metric.

28. The method of claim 1, further comprising enabling the medical care provider to adjust a medical treatment for at least one patient in response to the transmitted programmed alert.

29. The method of claim 1, further comprising providing a reminder to the at least one patient over the automated phone system to conform to prescribed medical treatment, or inviting the at least one patient to ask a question to the medical care provider, or both.

30. The method of claim 1, wherein the automated phone system is configured to facilitate interactive communication between the medical care provider and at least one patient.
